(19) United States
(12) Patent Application Publication
(10) Pub. No.: US 2026/0193221 A1
(43) Pub. Date: Jul. 9, 2026

(54) SOLID FORMS COMPRISING (5R,6S)-10,11-DIFLUORO-12-((2-FLUORO-4-IODOPHENYL)AMINO)-5,6-DIHYDROXY-4,5,6,7-TETRAHYDRO-1H-SPIRO[BENZO[B][1,5,4]OXATHIAZECINE-3,1'-CYCLOPROPANE] 2,2-DIOXIDE, AND COMPOSITIONS COMPRISING AND METHODS OF USING THE SAME

(71) Applicant: PASITHEA THERAPEUTICS CORP., Miami, FL (US)

(72) Inventors: Mathew LAZARUS, Mountain Lakes, NJ (US); Tiago Reis MARQUES, Miami, FL (US); Siyi JIANG, Shanghai (CN); Ming GAO, Shanghai (CN); Lele SUN, Shanghai (CN); Uday KHIRE, Orange, CT (US)

(73) Assignee: PASITHEA THERAPEUTICS CORP., Miami, FL (US)

(21) Appl. No.: 19/559,551

(22) Filed: Mar. 6, 2026

Related U.S. Application Data
(63) Continuation of application No. PCT/US2025/010064, filed on Jan. 2, 2025.
(60) Provisional application No. 63/722,522, filed on Nov. 19, 2024.

(30) Foreign Application Priority Data
Jan. 5, 2024 (WO) PCT/CN2024/070736

(51) Int. Cl. C07D 291/08; A61K 31/395; A61P 35/00

(52) U.S. Cl. CPC C07D 291/08; A61K 31/395; A61P 35/00

(57) ABSTRACT

Described herein are solid forms of (5R,6S)-10,11-difluoro-12-((2-fluoro-4-iodophenyl)amino)-5,6-dihydroxy-4,5,6, 7-tetrahydro-1H-spiro[benzo[b][1,5, 4]oxathiazecine-3,1′-cyclopropane]2,2-dioxide in amorphous and crystalline forms, and processes of preparation, and pharmaceutical compositions thereof. Also provided are methods of their use for treating, preventing, or ameliorating a disease or disorder in which the RAS pathway is upregulated such as cancer, RASopathies and laminopathies. Formula (I).

### 1. CROSS-REFERENCE TO RELATED APPLICATIONS

This application claims the benefit of U.S. Provisional Application No. 63/722,522, filed Nov. 19, 2024 and foreign application PCT/CN2024/070736, filed on Jan. 5, 2024, the disclosures of which are incorporated by reference herein in their entirety.

### 2. FIELD

Provided herein are solid forms comprising (5R,6S)-10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide. Pharmaceutical compositions comprising such solid forms and methods of use of such solid forms for treating, preventing, and managing various disorders are also provided herein.

### 3. BACKGROUND

Mitogen-activated protein kinase (MAPK) is relevant to many cancers. MAPKs specifically phosphorylate serine/threonine residues of proteins, that are activated by a variety of external stimuli (for example, mitogens and growth factors) to manifest its actions inside the cell. The activation of MAPKs regulates many functions of the cells with physiological implications such as cell growth, survival, apoptosis, differentiation, proliferation and gene expression. (G. Pearson, et al., Endocr: Rev., 2001, 153-183.)

MEK1 and 2 are two human kinases in the middle of the MAPK-cascade involving upstream Rat Sarcoma Virus (RAS)-Rapidly accelerated Fibrosarcoma (RAF) and downstream extracellular regulated kinases (ERK)s. This signal transduction cascade resulting in phosphorylation of ERKs is extensively studied in cancer pathology. Human cancers frequently harbor mutations in RAS oncogene family members, which drive oncogenesis by increasing cellular proliferation and survival. These are small protein GTPases, regulated by a switch between active GTP-linked and inactive GDP-bound states that is governed by a complex network of guanine exchange factors (GEFs, favoring RAS-GTP) and GTPase activating factors (GAPs, favoring RAS-GDP). (Cox A D, et al., Nat Rev Drug Discov. 2014; 13:828-51.) RAS activation either due to extrinsic recruitment by transmembrane tyrosine kinase receptors or intrinsic mutations propagates through the downstream RAF-MEK-ERK and PI3K-AKT signaling pathways. Besides RAS-activating mutations that confer independence from physiological regulators, human cancers harbor mutations in other RAS network genes such as NF1 (encoding neurofibromin, a RAS GAP), BRAF, or PTPN11 (encoding the SHP2 tyrosine phosphatase involved in RAS activation). (Decroocq J, et al., Leukemia (2022) 36:1237-1252.)

Several non-ATP-competitive, allosteric MEK1/2 inhibitors have been developed and assessed in clinical studies, primarily for cancers in which ERK1/2 signaling is aberrantly activated and three of them are FDA approved drugs. (Zhao Y, Adjei A A. Nat Rev Clin Oncol. 2014; 11:385; Caunt C J, Sale M J, Smith P D, Cook S J. Nat Rev Cancer. 2015; 15:577; Shang J, Lu S, Jiang Y, Zhang J. Chem Biol Drug Des. 2016; 88:485; Hao C, et al., Eu. J. Med. Chem. 2023, 251: 115236; Heinzerling L., ESMO Open 2019; 4:e000491.doi:10.1136/esmoopen-2019-000491; Stalnecker C A et al., Sci Signal. 2020, 13(624): 1-14; Echevarria-Vargas I M et al., Melanoma Manag. (2017) 4(4), 183-186; Wang A et al., IUBMB Life, 2013, 65(9):748-758.). These inhibitors are selective towards MEK1/2, as they bind to non-ATP-competitive allosteric sites. In addition to cancer, MEK1/2 inhibitors have potential to be useful in other diseases.

The compound chemically named 10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide belongs to a novel class of MEK1/2 inhibitors having a macrocyclic scaffold, which has been disclosed in U.S. Pat. No. 9,034,861, which is herein incorporated by reference in its entirety.

The identification and selection of a solid form of a pharmaceutical compound are complex, given that a change in solid form may affect a variety of physical and chemical properties, which may provide benefits or drawbacks in processing, formulation, stability, bioavailability, storage, handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical or chemical stability (see, e.g., S. R. Vippagunta et ac., Adv. Drug. Deliv. Rev., (2001) 48:3-26; L. Yu, Adv. Drug. Deliv. Rev., (2001) 48:27-42).

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound (see, e.g., S. R. Byrn et al., Solid State Chemistry of Drugs, (1999) SSCI, West Lafayette). The importance of discovering polymorphs was underscored by the case of Ritonavir™, an HIV protease inhibitor that was formulated as soft gelatin capsules. About two years after the product was launched, the unanticipated precipitation of a new, less soluble polymorph in the formulation necessitated the withdrawal of the product from the market until a more consistent formulation could be developed (see S. R. Chemburkar et al., Org. Process Res. Dev., (2000) 4:413-417).

Notably, it is not possible to predict apriori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (at present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable and marketable pharmaceutical product.

### 4. SUMMARY

Provided herein are solid forms (e.g., crystalline forms, amorphous forms, polymorphs or mixtures thereof) comprising Compound 1:

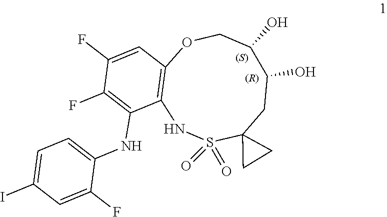

having the chemical name (5R,6S)-10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide. Also provided herein are methods of preparing, isolating, and characterizing the solid forms.

In one embodiment, the solid form comprises Compound 1. In one embodiment, the solid form is Form A, Form B, or Form C of a free base of Compound 1, as provided herein.

The solid forms provided herein are useful as active pharmaceutical ingredients for the preparation of formulations for use in animals or humans. Thus, embodiments herein encompass the use of these solid forms as a final drug product. Certain embodiments herein provide pharmaceutical compositions comprising a single-component crystal form, a multiple-component crystal form, a single-component amorphous form and/or a multiple-component amorphous form comprising Compound 1 and a pharmaceutically acceptable diluent, excipient or carrier.

Also provided are pharmaceutical compositions formulated for administration by an appropriate route and means containing effective concentrations of a solid form comprising Compound 1 provided herein, and optionally comprising at least one pharmaceutical carrier.

In one embodiment, the pharmaceutical compositions deliver amounts effective for the treatment of diseases or disorders in which the RAS pathway is upregulated such as cancer, RASopathies (e.g., Noonan syndrome, Costello syndrome, etc) and laminopathies (e.g., cardiomyopathies, muscular dystrophy, dysplasia, etc.). In one embodiment, the pharmaceutical compositions deliver amounts effective for the treatment and/or prevention of cancer, RASopathies and laminopathies. In one embodiment, the pharmaceutical compositions deliver amounts effective for the amelioration of cancer, RASopathies and laminopathies.

In one embodiment, provided herein are methods of treating diseases or disorders in which the RAS pathway is upregulated such as cancer, RASopathies and laminopathies comprising administering the solid forms or pharmaceutical compositions provided herein. Also provided herein are combination therapies using the solid forms or pharmaceutical compositions provided herein, in combination with a therapy, e.g., another pharmaceutical agent with activity against diseases or disorders in which the RAS pathway is upregulated such as cancer, RASopathies and laminopathies or their symptoms. Examples of therapies within the scope of the methods include, but are not limited to, surgery, chemotherapy, radiation therapy, biological therapy, stem cell transplantation, cell therapy, and combinations thereof.

These and other aspects of the subject matter described herein will become evident upon reference to the following detailed description.

### 5. BRIEF DESCRIPTION OF THE DRAWINGS

FIG. 1A provides a representative X-ray powder diffraction (XRPD) pattern of Form A of a free base of Compound 1. FIG. 1B provides a representative X-ray powder diffraction (XRPD) pattern of Form A of a free base of Compound 1 (without Kapton).

FIG. 2 provides a representative thermal gravimetric analysis (TGA) thermogram of Form A of a free base of Compound 1.

FIG. 3 provides a representative differential scanning calorimetry (DSC) thermogram of Form A of a free base of Compound 1.

FIG. 4 provides a representative dynamic vapor sorption (DVS) isotherm plot of Form A of a free base of Compound 1.

FIG. 5 provides a ¹H-NMR spectrum of Form A.

FIG. 6 provides a SEM microphoto of Form A.

FIG. 7 provides a representative XRPD pattern of Form B of a free base of Compound 1.

FIG. 8 provides a representative thermal gravimetric analysis (TGA) thermogram of Form B of a free base of Compound 1.

FIG. 9 provides a representative differential scanning calorimetry (DSC) thermogram of Form B of a free base of Compound 1.

FIG. 10 provides a ¹H-NMR spectrum of Form B.

FIG. 11 provides a representative XRPD pattern of Form C of a free base of Compound 1.

FIG. 12 provides a representative thermal gravimetric analysis (TGA) thermogram of Form C of a free base of Compound 1.

FIG. 13 provides a representative differential scanning calorimetry (DSC) thermogram of Form C of a free base of Compound 1.

FIG. 14 provides a ¹H-NMR spectrum of Form C.

FIG. 15 provides a representative XRPD pattern of amorphous form of a free base of Compound 1.

FIG. 16 provides a representative thermal gravimetric analysis (TGA) thermogram of amorphous form of a free base of Compound 1.

FIG. 17 provides a representative differential scanning calorimetry (DSC) thermogram of amorphous form of a free base of Compound 1.

FIG. 18 provides a ¹H-NMR spectrum of the amorphous form.

FIG. 19 provides an overlay of XRPD patterns of Form A, Form B, Form C, and amorphous form of Compound 1.

FIG. 20 provides an XRPD overlay of samples obtained from bulk stability experiments and the reference.

FIG. 21 provides a schematic diagram of interconversion relationship of polymorphs.

FIG. 22 provides an XRPD of an amorphous solid of an embodiment described herein.

FIG. 23 provides a PK plasma curve at day 1 of Compound 1 when tested in clinical trial setting.

FIG. 24 provides a PK plasma curve at day 22 of Compound 1 when tested in clinical trial setting.

### 6. DETAILED DESCRIPTION

### 6.1 Definitions

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein and unless otherwise specified, the terms “about” and “approximately,” when used in connection with a numeric value or a range of values which is provided to characterize a particular solid form, e.g., a specific temperature or temperature range, such as, for example, that describing a melting, dehydration, desolvation or glass transition temperature; a mass change, such as, for example, a mass change as a function of temperature or humidity; a solvent or water content, in terms of, for example, mass or a percentage; or a peak position, such as, for example, in analysis by IR or Raman spectroscopy or XRPD; indicate that the value or range of values may deviate to an extent deemed reasonable to one of ordinary skill in the art while still describing the particular solid form. For example, in particular embodiments, the terms “about” and “approximately,” when used in this context, indicate that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of XRPD peak position may vary by up to ±0.2 degrees 2θ while still describing the particular XRPD peak. As used herein, a tilde (i.e., “~”) preceding a numerical value or range of values indicates “about” or “approximately.”

Unless otherwise specified, the terms “X-ray powder diffraction”, “powder X-ray diffraction”, “PXRD”, and “XRPD” are used interchangeably in this application.

As used herein and unless otherwise specified, the terms “solid form” and related terms refer to a physical form which is not predominantly in a liquid or a gaseous state. As used herein, the terms “solid form” and “solid forms” encompass semi-solids. Solid forms may be crystalline, amorphous, partially crystalline, partially amorphous, or mixtures of forms.

The solid forms provided herein may have varying degrees of crystallinity or lattice order. The solid forms provided herein are not limited by any particular degree of crystallinity or lattice order, and may be 0-100% crystalline. Methods of determining the degree of crystallinity are known to those of ordinary skill in the, such as those described in Suryanarayanan, R., X-Ray Power Diffractometry, Physical Characterization of Pharmaceutical Salts, H. G. Brittain, Editor, Mercel Dekkter, Murray Hill, N.J., 1995, pp. 187-199, which is incorporated herein by reference in its entirety. In some embodiments, the solid forms provided herein are about 0, 5, 10, 15, 20, 25, 30, 35, 40, 45, 50, 55, 60, 65, 70, 75, 80, 85, 90, 95 or 100% crystalline.

As used herein and unless otherwise specified, the term “crystalline” and related terms used herein, when used to describe a substance, component, product, or form, mean that the substance, component, product, or form is substantially crystalline, for example, as determined by X-ray diffraction. See, e.g., Remington: The Science and Practice of Pharmacy, 21^{st} edition, Lippincott, Williams and Wilkins, Baltimore, MD (2005); The United States Pharmacopeia, 23^{rd} edition, 1843-1844 (1995).

As used herein and unless otherwise specified, the term “crystal form,” “crystal forms,” and related terms herein refer to solid forms that are crystalline. Crystal forms include single-component crystal forms and multiple-component crystal forms, and include, but are not limited to, polymorphs, solvates, hydrates, and other molecular complexes, as well as salts, solvates of salts, hydrates of salts, co-crystals of salts, other molecular complexes of salts, and polymorphs thereof. In certain embodiments, a crystal form of a substance may be substantially free of amorphous forms and/or other crystal forms. In certain embodiments, a crystal form of a substance may contain less than about 1%, 2%, 3%, 4%, 5%, 6%, 7%, 8%, 9%, 10%, 15%, 20%, 25%, 30%, 35%, 40%, 45% or 50% of one or more amorphous form(s) and/or other crystal form(s) on a weight basis. In certain embodiments, a crystal form of a substance may be physically and/or chemically pure. In certain embodiments, a crystal form of a substance may be about 99%, 98%, 97%, 96%, 95%, 94%, 93%, 92%, 91% or 90% physically and/or chemically pure.

A “single-component” solid form comprising a compound consists essentially of the compound. A “multiple-component” solid form comprising a compound comprises a significant quantity of one or more additional species, such as ions and/or molecules, within the solid form. For example, in certain embodiments, a crystalline multiple-component solid form comprising a compound further comprises one or more species non-covalently bonded at regular positions in the crystal lattice. For another example, in certain embodiments, an amorphous multiple-component solid form comprising a compound further comprises one or more polymer(s), and the compound is dispersed in a solid matrix that comprises the polymer(s).

Crystal forms of a substance may be obtained by a number of methods. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding, and solvent-drop grinding.

Unless otherwise specified, the terms “polymorph,” “polymorphic form,” “polymorphs,” “polymorphic forms,” and related terms herein refer to two or more crystal forms that consist essentially of the same molecule, molecules or ions. Like different crystal forms, different polymorphs may have different physical properties, such as, for example, melting temperatures, heats of fusion, solubilities, dissolution rates, and/or vibrational spectra as a result of a different arrangement or conformation of the molecules or ions in the crystal lattice. The differences in physical properties exhibited by polymorphs may affect pharmaceutical parameters, such as storage stability, compressibility and density (important in formulation and product manufacturing), and dissolution rate (an important factor in bioavailability). Differences in stability can result from changes in chemical reactivity (e.g., differential oxidation, such that a dosage form discolors more rapidly when comprised of one polymorph than when comprised of another polymorph) or mechanical changes (e.g., tablets crumble on storage as a kinetically favored polymorph converts to thermodynamically a more stable polymorph) or both (e.g., tablets of one polymorph are more susceptible to breakdown at high humidity). As a result of solubility/dissolution differences, in the extreme case, some polymorphic transitions may result in lack of potency or, at the other extreme, toxicity. In addition, the physical properties of the crystal may be important in processing (for example, one polymorph might be more likely to form solvates or might be difficult to filter and wash free of impurities, and particle shape and size distribution might be different between polymorphs).

As used herein and unless otherwise specified, the term “amorphous,” “amorphous form,” and related terms used herein, mean that the substance, component or product in question is not substantially crystalline as determined by X-ray diffraction. In particular, the term “amorphous form” describes a disordered solid form, i.e., a solid form lacking long range crystalline order. In certain embodiments, an amorphous form of a substance may be substantially free of other amorphous forms and/or crystal forms. In other embodiments, an amorphous form of a substance may contain less than about 1%, 2%, 3%, 4%, 5%, 10%, 15%, 20%, 25%, 30%, 35%, 40%, 45% or 50% of one or more other amorphous forms and/or crystal forms on a weight basis. In certain embodiments, an amorphous form of a substance may be physically and/or chemically pure. In certain embodiments, an amorphous form of a substance may be about 99%, 98%, 97%, 96%, 95%, 94%, 93%, 92%, 91% or 90% physically and/or chemically pure. In certain embodiments, an amorphous form of a substance may comprise additional components or ingredients (for example, an additive, a polymer, or an excipient that may serve to further stabilize the amorphous form). In certain embodiments, amorphous form may be a solid solution.

Amorphous forms of a substance can be obtained by a number of methods. Such methods include, but are not limited to, heating, melt cooling, rapid melt cooling, solvent evaporation, rapid solvent evaporation, desolvation, sublimation, grinding, ball-milling, cryo-grinding, spray drying, and freeze drying.

Unless otherwise specified, the terms “solvate” and “solvated,” as used herein, refer to a solid form of a substance which contains solvent. The terms “hydrate” and “hydrated” refer to a solvate wherein the solvent comprises water. “Polymorphs of solvates” refer to the existence of more than one solid form for a particular solvate composition. Similarly, “polymorphs of hydrates” refers to the existence of more than one solid form for a particular hydrate composition. The term “desolvated solvate,” as used herein, refers to a solid form of a substance which can be made by removing the solvent from a solvate. The terms “solvate” and “solvated,” as used herein, can also refer to a solvate of a salt, co-crystal, or molecular complex. The terms “hydrate” and “hydrated,” as used herein, can also refer to a hydrate of a salt, co-crystal, or molecular complex.

Techniques for characterizing crystal forms and amorphous forms include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis and Karl Fischer analysis. Characteristic unit cell parameters may be determined using one or more techniques such as, but not limited to, X-ray diffraction and neutron diffraction, including single-crystal diffraction and powder diffraction. Techniques useful for analyzing powder diffraction data include profile refinement, such as Rietveld refinement, which may be used, e.g., to analyze diffraction peaks associated with a single phase in a sample comprising more than one solid phase. Other methods useful for analyzing powder diffraction data include unit cell indexing, which allows one of skill in the art to determine unit cell parameters from a sample comprising crystalline powder.

In certain embodiments, the solid forms, e.g., crystal or amorphous forms, provided herein are substantially pure, i.e., substantially free of other solid forms and/or of other chemical compounds, containing less than about 25%, 20%, 15%, 10%, 9%, 8%, 7%, 6%, 5%, 4%, 3%, 2%, 1%, 0.75%, 0.5%, 0.25% or 0.1% percent by weight of one or more other solid forms and/or of other chemical compounds.

As used herein, and unless otherwise indicated, a chemical compound, solid form, or composition that is “substantially free” of another chemical compound, solid form, or composition means that the compound, solid form, or composition contains, in certain embodiments, less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2% 0.1%, 0.05%, or 0.01% by weight of the other compound, solid form, or composition.

As used herein, and unless otherwise specified, a solid form that is “substantially physically pure” is substantially free from other solid forms. In certain embodiments, a crystal form that is substantially physically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4% 0.3% 0.2%, 0.1%, 0.05%, or 0.01% of one or more other solid forms on a weight basis. The detection of other solid forms can be accomplished by any method apparent to a person of ordinary skill in the art, including, but not limited to, diffraction analysis, thermal analysis, elemental combustion analysis and/or spectroscopic analysis.

As used herein, and unless otherwise specified, a solid form that is “substantially chemically pure” is substantially free from other chemical compounds (i.e., chemical impurities). In certain embodiments, a solid form that is substantially chemically pure contains less than about 50%, 45%, 40%, 35%, 30%, 25%, 20%, 15%, 10%, 9%, 8%, 7%, 6%, 5%, 4%, 3%, 2%, 1%, 0.5%, 0.4%, 0.3%, 0.2%, 0.1%, 0.05%, or 0.01% of one or more other chemical compounds on a weight basis. The detection of other chemical compounds can be accomplished by any method apparent to a person of ordinary skill in the art, including, but not limited to, methods of chemical analysis, such as, e.g., mass spectrometry analysis, spectroscopic analysis, thermal analysis, elemental combustion analysis and/or chromatographic analysis.

Solid forms may exhibit distinct physical characterization data that are unique to a particular solid form, such as the crystal forms provided herein. These characterization data may be obtained by various techniques known to those skilled in the art, including for example X-ray powder diffraction, differential scanning calorimetry, thermal gravimetric analysis, and nuclear magnetic resonance spectroscopy. The data provided by these techniques may be used to identify a particular solid form. One skilled in the art can determine whether a solid form is one of the forms provided herein by performing one of these characterization techniques and determining whether the resulting data “matches” the reference data provided herein, which is identified as being characteristic of a particular solid form. Characterization data that “matches” those of a reference solid form is understood by those skilled in the art to correspond to the same solid form as the reference solid form. In analyzing whether data “match,” a person of ordinary skill in the art understands that particular characterization data points may vary to a reasonable extent while still describing a given solid form, due to, for example, experimental error and routine sample-to-sample analysis variation.

As used herein and unless otherwise indicated, the terms “substantially pure” when applied to a chiral compound or “stereoisomer” or “substantially stereomerically pure” means one stereoisomer of a compound disclosed herein that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein have chiral centers and can occur as individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

As used herein, and unless otherwise specified, the term “pharmaceutically acceptable salts” refers to salts prepared from pharmaceutically acceptable, relatively non-toxic acids, including inorganic acids and organic acids. In certain embodiments, suitable acids include, but are not limited to, acetic, adipic, 4-aminosalicylic, ascorbic, aspartic, benzenesulfonic, benzoic, camphoric, camphorsulfonic, capric, caproic, caprylic, cinnamic, carbonic, citric, cyclamic, dihydrogenphosphoric, 2,5-dihydroxybenzoic (gentisic), 1,2-ethanedisulfonic, ethanesulfonic, fumaric, galactunoric, gluconic, glucuronic, glutamic, glutaric, glycolic, hippuric, hydrobromic, hydrochloric, hydriodic, isobutyric, isethionic, lactic, maleic, malic, malonic, mandelic, methanesulfonic, monohydrogencarbonic, monohydrogen-phosphoric, monohydrogensulfuric, mucic, 1,5-naphthalenedisulfonic, nicotinic, nitric, oxalic, pamoic, pantothenic, phosphoric, phthalic, propionic, pyroglutamic, salicylic, suberic, succinic, sulfuric, tartaric, toluenesulfonic acid, and the like (see, e.g., S. M. Berge et al., J. Pharm. Sci., 66:1-19 (1977); and Handbook of Pharmaceutical Salts: Properties, Selection and Use, P. H. Stahl and C. G. Wermuth, Eds., (2002), Wiley, Weinheim). In certain embodiments, suitable acids are strong acids (e.g., with pKa less than about 1), including, but not limited to, hydrochloric, hydrobromic, sulfuric, nitric, methanesulfonic, benzene sulfonic, toluene sulfonic, naphthalene sulfonic, naphthalene disulfonic, pyridine-sulfonic, or other substituted sulfonic acids. Also included are salts of other relatively non-toxic compounds that possess acidic character, including amino acids, such as aspartic acid and the like, and other compounds, such as aspirin, ibuprofen, saccharin, and the like. Acid addition salts can be obtained by contacting the neutral form of a compound with a sufficient amount of the desired acid, either neat or in a suitable solvent. As solids, salts can exist in crystalline or amorphous forms, or mixtures thereof. Salts can also exist in polymorphic forms.

As used herein and unless otherwise indicated, the terms “treat,” “treating” and “treatment” refer to alleviating or reducing the severity of a symptom associated with the disease or condition being treated, for example, cancer.

The term “prevention” includes the inhibition of a symptom of the particular disease or disorder, for example cancer. Generally, the term “preventing” refers to administration of the drug prior to the onset of symptoms, particularly to patients at risk of cancer.

As used herein and unless otherwise indicated, the term “managing” encompasses preventing the recurrence of the particular disease or disorder, in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein, “subject” or “patient” is an animal, typically a mammal, including a human, such as a human patient.

As used herein, and unless otherwise specified, the terms “therapeutically effective amount” and “effective amount” of a compound refer to an amount sufficient to provide a therapeutic benefit in the treatment, prevention and/or management of a disease, or to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

The terms “co-administration” and “in combination with” include the administration of one or more therapeutic agents (for example, a compound provided herein and another cancer agent or supportive care agent) either simultaneously, concurrently or sequentially with no specific time limits. In one embodiment, the agents are present in the cell or in the patient's body at the same time or exert their biological or therapeutic effect at the same time. In one embodiment, the therapeutic agents are in the same composition or unit dosage form. In another embodiment, the therapeutic agents are in separate compositions or unit dosage forms.

The term “supportive care agent” refers to any substance that treats, prevents or manages an adverse effect from treatment with Compound 1, or tautomers, or a pharmaceutically acceptable salt thereof.

In the context of a cancer, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP, as used herein, means the time from treatment onset until tumor progression; TTP does not include deaths. In one embodiment, PFS means the time from treatment onset until tumor progression or death. In one embodiment, PFS means the time from the first dose of compound to the first occurrence of disease progression or death from any cause. In one embodiment, PFS rates will be computed using the Kaplan-Meier estimates. Event-free survival (EFS) means the time from treatment onset until any treatment failure, including disease progression, treatment discontinuation for any reason, or death. In one embodiment, overall response rate (ORR) means the percentage of patients who achieve a response. In one embodiment, ORR means the sum of the percentage of patients who achieve complete and partial responses. In one embodiment, ORR means the percentage of patients whose best response≥partial response (PR), according to the IMWG Uniform Response Criteria. In one embodiment, duration of response (DoR) is the time from achieving a response until relapse or disease progression. In one embodiment, DoR is the time from achieving a response≥partial response (PR) until relapse or disease progression. In one embodiment, DoR is the time from the first documentation of a response until to the first documentation of progressive disease or death. In one embodiment, DoR is the time from the first documentation of a response≥partial response (PR) until to the first documentation of progressive disease or death. In one embodiment, time to response (TTR) means the time from the first dose of compound to the first documentation of a response. In one embodiment, TTR means the time from the first dose of compound to the first documentation of a response & partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

The term “RASopathies” refers to a group of syndromes, also called conditions or disorders, caused by changes in genes that send signals across the Ras/mitogen-activated protein kinase (Ras/MAPK) pathway. While there are different RASopathies (currently, fewer than 10 disorders) and each syndrome has unique features, some common features include heart defects, skin, bone, eye and muscle problems, short stature, learning problems, differences in appearance and an increased risk of developing benign and cancerous tumors. RASopathies include, but are not limited to, Noonan Syndrome, Costello Syndrome, Cardiofaciocutaneous Syndrome, Legius Syndrome, Capillary Arteriovenous Malformation Syndrome, Hereditary gingival fibromatosis, SYNGAP1 syndrome, Leopard Syndrome, neurofibromatosis 1 (NF1), neurofibromatosis 2 (NF2) (see, National Cancer Institute: https://dceg.cancer.gov/research/what-we-study/rasopathies).

The term “Laminopathies” refers to a group of rare genetic disorders caused by mutations in genes encoding proteins of the nuclear lamina. They are included in the more generic term nuclear envelopathies that refers to diseases associated with defects of the nuclear envelope. Laminopathies and other nuclear envelopathies have a large variety of clinical symptoms including skeletal and/or cardiac muscular dystrophy, lipodystrophy and diabetes, dysplasia, dermo- or neuropathy, leukodystrophy, and progeria (premature aging). Most of these symptoms develop after birth, typically during childhood or adolescence. Some laminopathies however may lead to an early death, and mutations of lamin B1 (LMNB1 gene) may be lethal before or at birth. Types of known laminopathies and other nuclear envelopathies include, but are not limited to, Atypical Werner syndrome, Buschke-Ollendorff syndrome, Cardiomyopathy, Charcot-Marie-Tooth disease, Emery-Dreifuss muscular dystrophies, Greenberg dysplasia, Hutchinson-Gilford progeria syndrome (HGPS), Leukodystrophy, demyelinating, adult-onset, autosomal dominant (ADLD), Limb-girdle muscular dystrophy type 1B (LGMD1B), Lipoatrophy with diabetes, hepatic steatosis, hypertrophic cardiomyopathy, and leukomelanodermic papules (LDHCP), Mandibuloacral dysplasia, Pelger-Huet anomaly (PHA), Restrictive dermopathy.

The term “Neurofibromatosis” or “Neurofibromatoses” refers to a group of genetic disorders, belonging to RASopathies as mentioned above, that cause tumors to form on nerve tissue. These tumors can develop anywhere in the nervous system, including the brain, spinal cord and nerves. There are three types of neurofibromatosis: neurofibromatosis 1 (NF1), neurofibromatosis 2 (NF2) and schwannomatosis. NF1 is usually diagnosed in childhood, while NF2 and schwannomatosis are usually diagnosed in early adulthood. The tumors in these disorders are usually noncancerous (benign), but sometimes can become cancerous (malignant). Symptoms are often mild. However, complications of neurofibromatosis can include hearing loss, learning impairment, heart and blood vessel (cardiovascular) problems, loss of vision, and severe pain In certain embodiments, a subject who is diagnosed with NF1 has cutaneous and/or plexiform neurofibromas.

Unless otherwise specified, to the extent that there is a discrepancy between a depicted chemical structure of a compound provided herein and a chemical name of a compound provided herein, the chemical structure shall control.

### Solid Forms of Compound 1

In certain embodiments, provided herein are solid forms of Compound 1. In certain embodiments, the solid form is crystalline. In certain embodiments, the solid form is a single-component solid form. In certain embodiments, the solid form is an anhydrate. In certain embodiments, the solid form is a solvate.

While not intending to be bound by any particular theory, certain solid forms are characterized by physical properties, e.g., stability, solubility and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy and thermal analysis), as described herein and known in the art.

The solid forms provided herein (e.g., Form A, Form B, Form C, and the amorphous solid of Compound 1) may be characterized using a number of methods known to a person skilled in the art, including, but not limited to, single crystal X-ray diffraction, X-ray powder diffraction (XRPD), microscopy (e.g., scanning electron microscopy (SEM)), thermal analysis (e.g., differential scanning calorimetry (DSC), dynamic vapor sorption (DVS), thermal gravimetric analysis (TGA), and hot-stage microscopy), spectroscopy (e.g., infrared, Raman, and solid-state nuclear magnetic resonance), ultra-high performance liquid chromatography (UHPLC), and proton nuclear magnetic resonance (¹H-NMR) spectrum. The particle size and size distribution of the solid form provided herein may be determined by conventional methods, such as laser light scattering technique.

The purity of the solid forms provided herein may be determined by standard analytical methods, such as thin layer chromatography (TLC), gel electrophoresis, gas chromatography, ultra-high performance liquid chromatography (UHPLC), and mass spectrometry (MS).

It should be understood that the numerical values of the peaks of an X-ray powder diffraction pattern may vary slightly from one machine to another or from one sample to another, and so the values quoted are not to be construed as absolute, but with an allowable variability, such as 10.2° 20 (see United State Pharmacopoeia, page 2228 (2003)), unless indicated otherwise herein.

### Form A

In certain embodiments, provided herein is Form A.

In one embodiment, Form A is a solid form of Compound 1. In one embodiment, Form A is a solid form of a free base of Compound 1. In another embodiment, Form A is crystalline. In one embodiment, Form A is an anhydrate solid form of Compound 1. In one embodiment, Form A is an anhydrate solid form of a free base of Compound 1.

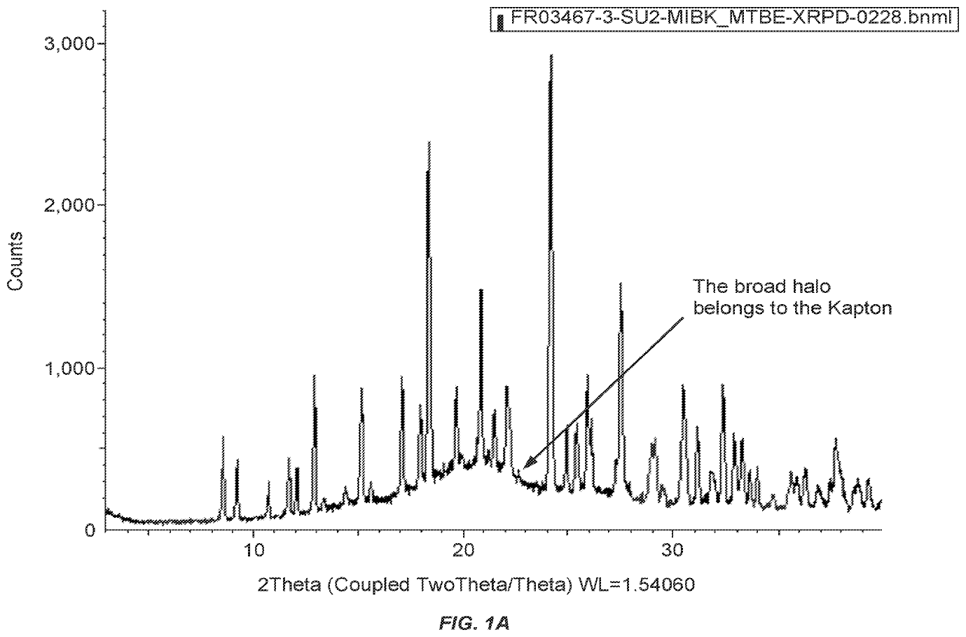

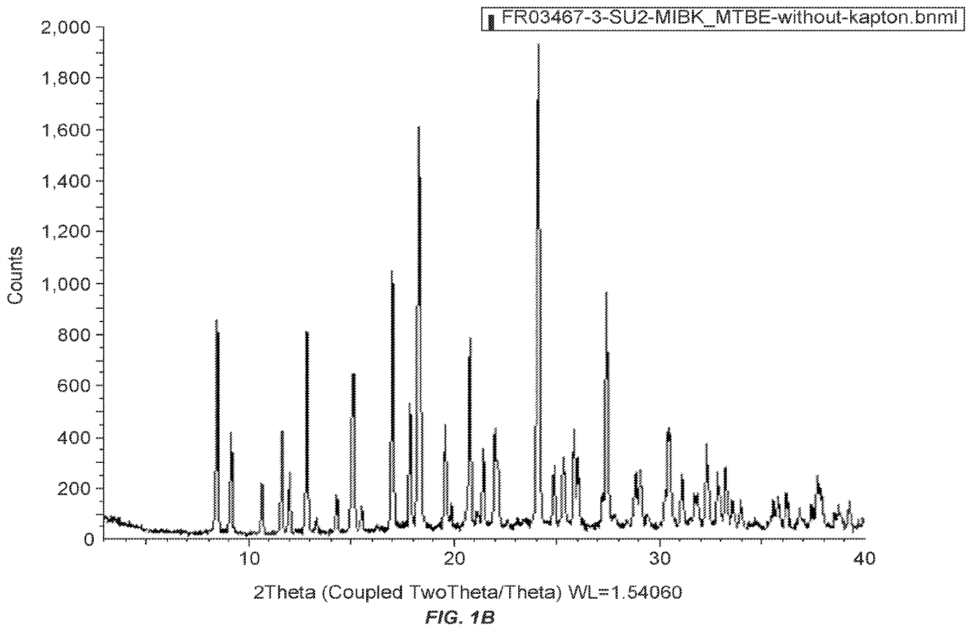

A representative XRPD pattern of Form A is provided in FIGS. 1A and 1B.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, or all of the peaks located at approximately the following positions: 8.5, 9.2, 10.7, 11.6, 12.0, 12.9, 13.3, 14.3, 15.1, 15.5, 16.3, 17.02, 17.9, 18.3, 19.6, 19.9, 20.8, 21.1, 21.4, 22.0, 22.6, 23.09, 24.1, 24.9, 25.3, 25.8, 26.0, 27.4, 27.8, 28.8, 29.1, 29.4, 30.4, 31.1, 31.5, 31.9, 32.3, 32.8, 33.2, 33.6, 33.9, 34.7, 35.6, 35.8, 36.2, 36.8, 37.4, 37.7, 38.5, 38.7, and 39.2° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by 15 of the peaks. In one embodiment, the solid form is characterized by 25 of the peaks. In one embodiment, the solid form is characterized by all of the peaks.

In one embodiment, provided herein is a solid form comprising a free base of Compound 1, characterized by an XRPD pattern comprising peaks at approximately 8.5, 15.1, and 18.3° 2θ. In one embodiment, the XRPD pattern further comprises peaks at approximately 9.2, 12.0, and 17.0° 2θ. In one embodiment, the XRPD pattern further comprises peaks at approximately 20.8, 22.0, 24.1, and 27.4° 2θ. In one embodiment, the XRPD pattern comprises peaks at approximately 8.5, 9.2, 10.7, 11.6, 12.0, 12.8, 15.1, 17.0, 18.3, 20.8, 22.0, 24.1, and 27.4° 2θ.

In one embodiment, provided herein is a solid form comprising a free base of Compound 1, characterized by an XRPD pattern substantially as shown in FIG. 1A. In one embodiment, provided herein is a solid form comprising a free base of Compound 1, characterized by an XRPD pattern substantially as shown in FIG. 1B.

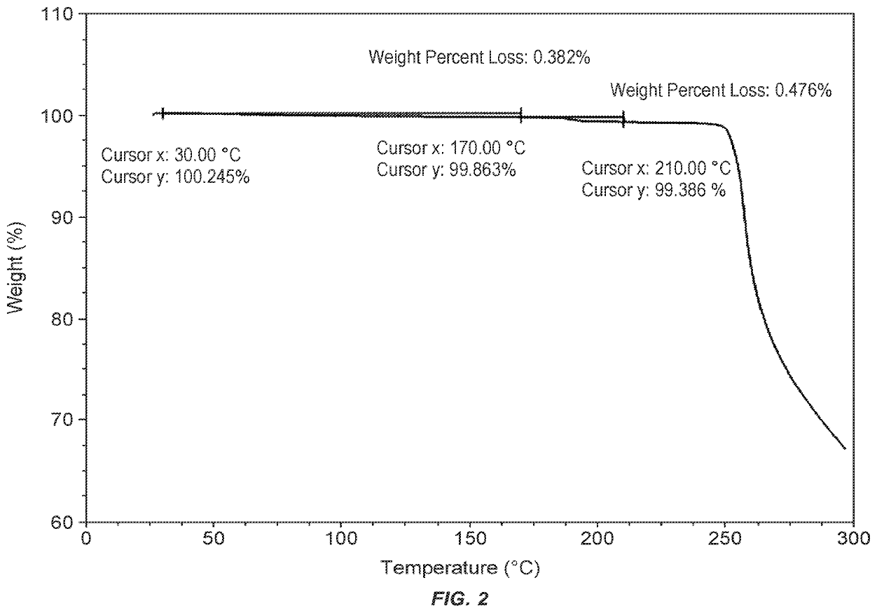

A representative thermal gravimetric analysis (TGA) thermogram of Form A is provided in FIG. 2. In one embodiment, provided herein is a solid form comprising a free base of Compound 1, which exhibits a thermal gravimetric analysis plot comprising two mass losses of about 0.4% and about 0.5% when heated from about 30° C. to about 260° C. In one embodiment, the solid form comprising a free base of Compound 1 exhibits about 0.4% mass loss when heated from about room temperature to about 170° C. and 0.5% mass loss when heated from about 170° C. to about 210° C. In one embodiment, the solid form comprising a free base of Compound 1 is an anhydrate. In one embodiment, the solid Form A comprising a free base of Compound 1 is an anhydrate. In one embodiment, provided herein is a solid form comprising a free base of Compound 1, characterized by a TGA thermogram substantially as shown in FIG. 2.

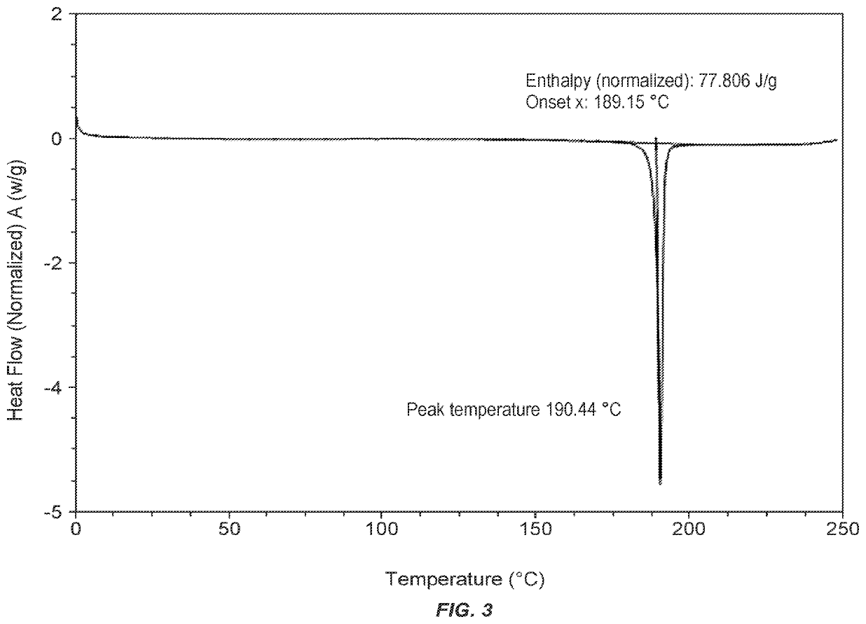

A representative differential scanning calorimetry (DSC) thermogram of Form A is presented in FIG. 3. In one embodiment, provided herein is a solid form comprising a free base of Compound 1, which exhibits, as characterized by DSC, a thermal event with an onset temperature of about 176° C. and a second thermal event with an onset temperature of about 186° C. In one embodiment, the first thermal event also has a peak temperature of about 178° C., and the second thermal event also has a peak temperature of about 189° C. In one embodiment, without being limited by any particular theory, the second thermal event corresponds to melting of Form A. In one embodiment, provided herein is a solid form comprising a free base of Compound 1, characterized by a DSC thermogram substantially as shown in FIG. 3.

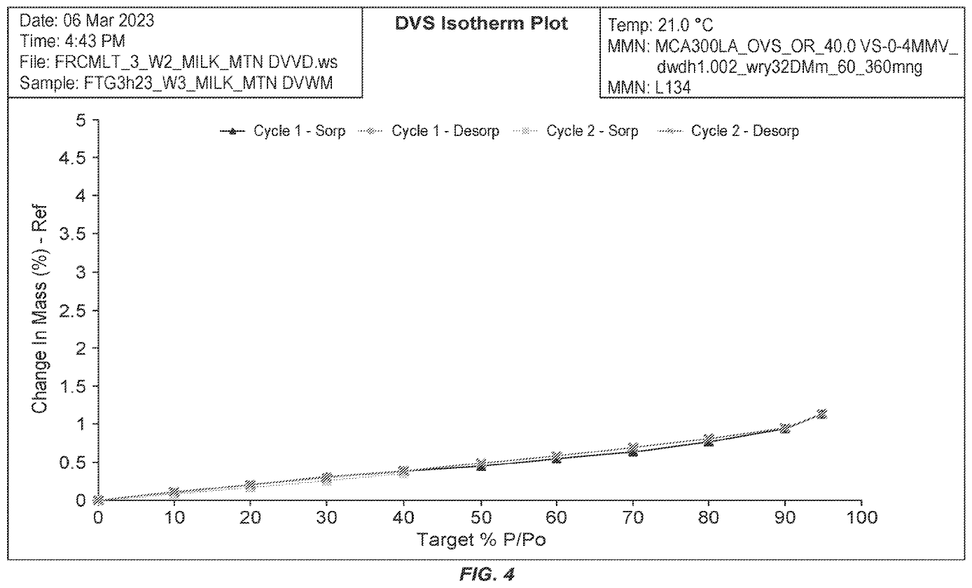

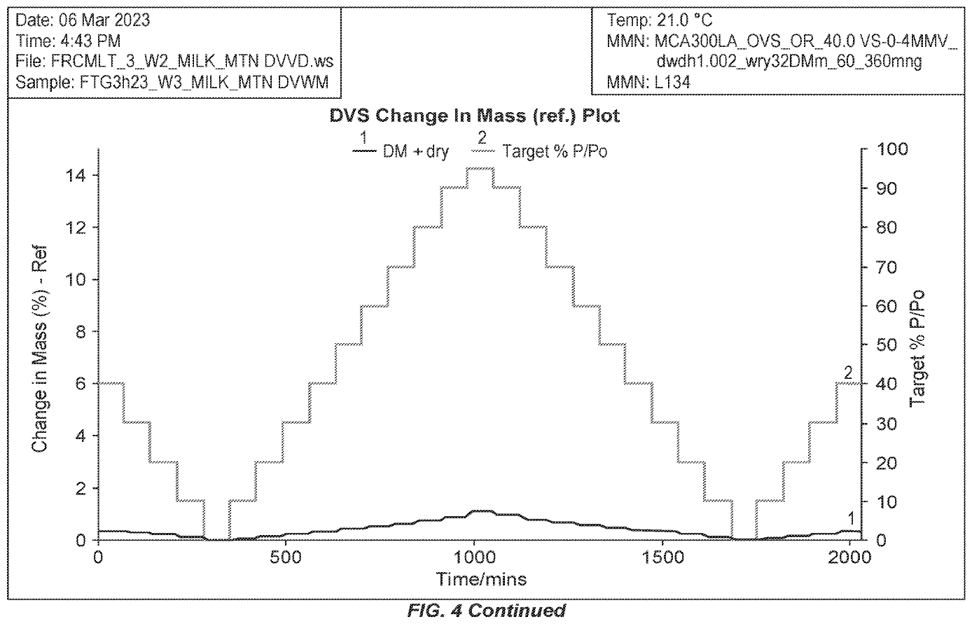

A representative dynamic vapor sorption (DVS) isotherm plot of the Form A is provided in FIG. 4. In one embodiment, provided herein is a solid form comprising a free base of Compound 1, which exhibits a mass increase of about 0.4% when subjected to an increase in a relative humidity (RH) from about 40% to about 80%. In one embodiment, provided herein is a solid form comprising a free base of Compound 1, characterized by a DVS isotherm plot substantially as shown in FIG. 4. In one embodiment, Form A is non hygroscopic. In one embodiment, Form A is slightly hygroscopic at high relative humidity.

In one embodiment, Form A of a free base of Compound 1 is prepared as discussed herein in section 5.2.

In one embodiment, provided herein is a solid form comprising Form A of a free base of Compound 1 and the amorphous form of a free base of Compound 1 provided herein. In one embodiment, provided herein is a solid form comprising Form A of a free base Compound 1 and one or more other crystalline forms of a free base of Compound 1 provided herein.

In another embodiment, Form A is substantially pure. In another embodiment, Form A is substantially physically pure. In certain embodiments, the substantially physically pure Form A is substantially free of other solid forms, e.g., amorphous solid. In certain embodiments, the purity of the substantially physically pure Form A is no less than about 95%, no less than about 96%, no less than about 97%, no less than about 98%, no less than about 98.5%, no less than about 99%, no less than about 99.5%, or no less than about 99.8%.

In certain embodiments, the composition comprising Form A is substantially physically pure. In certain embodiments, the composition comprising Form A is substantially free from other solid forms, for example, as containing no detectable amount of another solid form (e.g., another polymorph such as Form B or Form C). In certain embodiments, the composition comprising Form A contains no detectable amount of another solid form (e.g., another polymorph such as Form B or Form C), as determined by observing no detectable differences in an XRPD and/or DSC pattern between a single Form A crystal and the crystalline composition of Compound 1 ((5R,6S)-10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide).

### Form B

In certain embodiments, provided herein is Form B.

In one embodiment, Form B is a solid form of Compound 1. In one embodiment, Form B is a solid form of a free base of Compound 1. In another embodiment, Form B is crystalline. In one embodiment, Form B is a solvate solid form of Compound 1. In one embodiment, Form B is a solvate form of a free base of Compound 1.

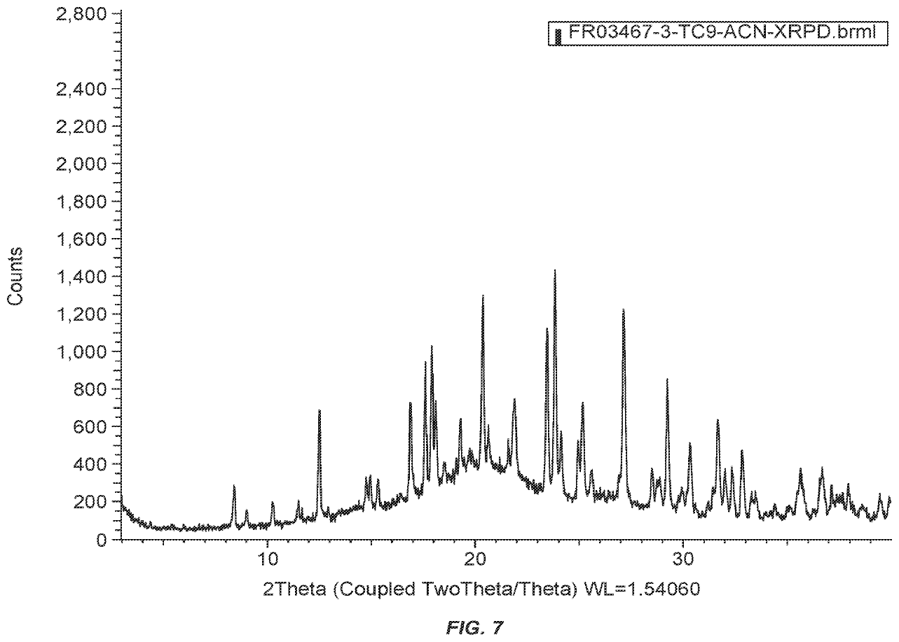

A representative XRPD pattern of Form B is provided in FIG. 7.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, or all of the peaks located at approximately the following positions: 8.4, 9.0, 10.3, 11.5, 12.5, 12.9, 14.8, 14.9, 15.3, 16.5, 16.9, 17.6, 17.9, 18.1, 18.5, 19.3, 19.9, 20.4, 20.6, 21.6, 21.9, 23.5, 23.8, 24.1, 24.9, 25.2, 25.6, 26.2, 26.9, 27.1, 28.5, 28.9, 29.2, 29.9, 30.3, 31.2, 31.4, 31.7, 32.0, 32.4, 32.8, 33.3, 33.5, 34.4, 35.1, 35.7, 36.7, 37.1, 37.6, 37.9, 38.1, 38.6, and 39.5° 2θ. In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, or all of the peaks located at approximately the following positions: 8.42, 9.02, 10.28, 11.51, 12.52, 12.94, 14.78, 14.95, 15.34, 16.47, 16.91, 17.62, 17.93, 18.11, 18.53, 19.30, 19.87, 20.37, 20.64, 21.61, 21.91, 23.46, 23.85, 24.15, 24.96, 25.18, 25.59, 26.19, 26.93, 27.16, 28.51, 28.87, 29.25, 29.94, 30.34, 31.20, 31.45, 31.68, 32.02, 32.36, 32.84, 33.31, 33.49, 34.42, 35.14, 35.66, 36.70, 37.15, 37.58, 37.94, 38.16, 38.61, and 39.47° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by 15 of the peaks. In one embodiment, the solid form is characterized by 25 of the peaks. In one embodiment, the solid form is characterized by all of the peaks.

In one embodiment, provided herein is a solid form comprising a free base of Compound 1, characterized by an XRPD pattern comprising peaks at approximately 8.4, 12.5, and 16.9° 2θ. In one embodiment, the XRPD pattern further comprises peaks at approximately 17.6, 17.9, and 18.1° 2θ. In one embodiment, the XRPD pattern further comprises peaks at approximately 20.4, 23.5, and 23.8° 2θ. In one embodiment, the XRPD pattern further comprises peaks at approximately 25.2, 27.1, and 29.2° 2θ. In one embodiment, the XRPD pattern comprises peaks at approximately 16.9, and 17.9° 2θ. In one embodiment, the XRPD pattern comprises peaks at approximately 16.9, 17.6, 17.9, and 18.1° 2θ. In one embodiment, the XRPD pattern comprises peaks at approximately 14.8, 14.9, and 15.3° 2θ. In one embodiment, the XRPD pattern comprises peaks at approximately 21.9, 23.5, 23.82° 2θ. In one embodiment, the XRPD pattern comprises peaks at approximately 8.4, 9.0, 10.3, 11.5, 12.5, 14.8, 14.9, 15.3, 16.9, 17.6, 17.9, 18.1, 19.3, 19.9, 20.4, 21.9, 23.5, 23.8, 24.1, 24.9, 25.2, 25.6, 27.1, 28.5, 28.9, 29.2, 30.3, 31.7, 32.0, 32.4, 32.8, 35.7, and 36.7° 2θ.

In one embodiment, provided herein is a solid form comprising a free base of Compound 1, characterized by an XRPD pattern comprising peaks at approximately 8.4, 12.5, and 16.9° 2θ±0.04° 2θ. In one embodiment, the XRPD pattern further comprises peaks at approximately 17.6, 17.9, and 18.1° 2θ±0.04° 2θ. In one embodiment, the XRPD pattern further comprises peaks at approximately 20.4, 23.5, and 23.8° 2θ±0.04° 2θ. In one embodiment, the XRPD pattern further comprises peaks at approximately 25.2, 27.1, and 29.2° 2θ±0.04° 2θ. In one embodiment, the XRPD pattern comprises peaks at approximately 16.91, and 17.93° 2θ±0.04° 2θ. In one embodiment, the XRPD pattern comprises peaks at approximately 16.91, 17.62, 17.93, and 18.11° 2θ±0.04° 2θ. In one embodiment, the XRPD pattern comprises peaks at approximately 14.78, 14.95, and 15.34° 2θ±0.04° 2θ. In one embodiment, the XRPD pattern comprises peaks at approximately 21.91, 23.46, 23.85° 2θ±0.04° 2θ. In one embodiment, the XRPD pattern comprises peaks at approximately 8.4, 9.0, 10.3, 11.5, 12.5, 14.8, 14.9, 15.3, 16.9, 17.6, 17.9, 18.1, 19.3, 19.9, 20.4, 21.9, 23.5, 23.8, 24.1, 24.9, 25.2, 25.6, 27.1, 28.5, 28.9, 29.2, 30.3, 31.7, 32.0, 32.4, 32.8, 35.7, and 36.7±0.04° 2θ.

In one embodiment, provided herein is a solid form comprising a free base of Compound 1, characterized by an XRPD pattern comprising peaks at approximately 8.4, 12.5, and 16.9° 2θ±0.02° 2θ. In one embodiment, the XRPD pattern further comprises peaks at approximately 17.6, 17.9, and 18.1° 2θ±0.02° 2θ. In one embodiment, the XRPD pattern further comprises peaks at approximately 20.4, 23.5, and 23.8° 2θ±0.02° 2θ. In one embodiment, the XRPD pattern further comprises peaks at approximately 25.2, 27.1, and 29.2° 2θ±0.02° 2θ. In one embodiment, the XRPD pattern comprises peaks at approximately 16.91, and 17.93° 2θ±0.02° 2θ. In one embodiment, the XRPD pattern comprises peaks at approximately 16.91, 17.62, 17.93, and 18.11° 2θ±0.02° 2θ. In one embodiment, the XRPD pattern comprises peaks at approximately 14.78, 14.95, and 15.34° 2θ±0.02° 2θ. In one embodiment, the XRPD pattern comprises peaks at approximately 21.91, 23.46, 23.85° 2θ±0.02° 2θ. In one embodiment, the XRPD pattern comprises peaks at approximately 8.4, 9.0, 10.3, 11.5, 12.5, 14.8, 14.9, 15.3, 16.9, 17.6, 17.9, 18.1, 19.3, 19.9, 20.4, 21.9, 23.5, 23.8, 24.1, 24.9, 25.2, 25.6, 27.1, 28.5, 28.9, 29.2, 30.3, 31.7, 32.0, 32.4, 32.8, 35.7, and 36.7±0.02° 2θ.

In one embodiment, provided herein is a solid form form comprising a free base of Compound 1, characterized by an XRPD pattern substantially as shown in FIG. 7.

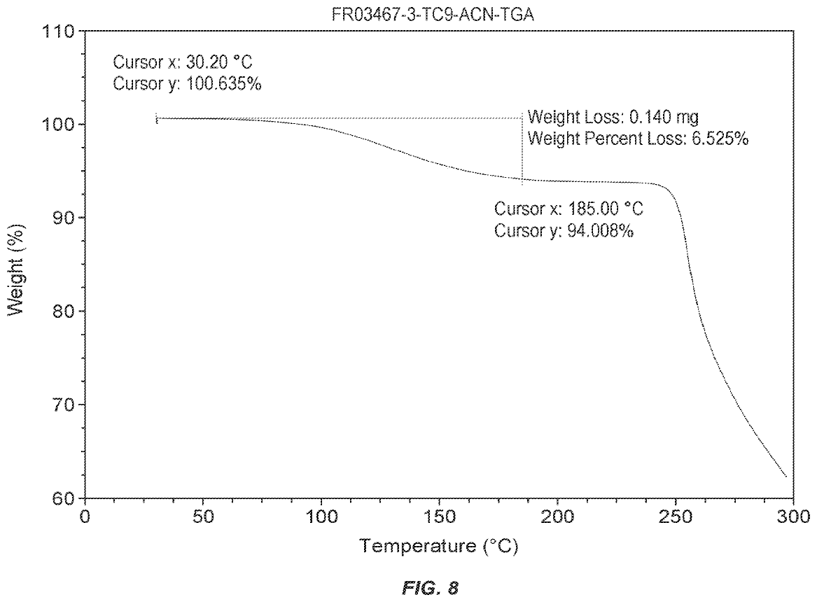

A representative thermal gravimetric analysis (TGA) thermogram of Form B is provided in FIG. 8. In one embodiment, provided herein is a solid form comprising a free base of Compound 1, which exhibits a thermal gravimetric analysis plot comprising a mass loss of about 6.5% when heated from about 30° C. to about 260° C. In one embodiment, without being limited by any particular theory, the weight loss corresponds to the loss of acetonitrile. In one embodiment, the solid form comprising a free base of Compound 1 is a solvate. In one embodiment, the the solid Form B comprising a free base of Compound 1 is an acetonitrile solvate. In one embodiment, provided herein is a solid form comprising a free base of Compound 1, characterized by a TGA thermogram substantially as shown in FIG. 8.

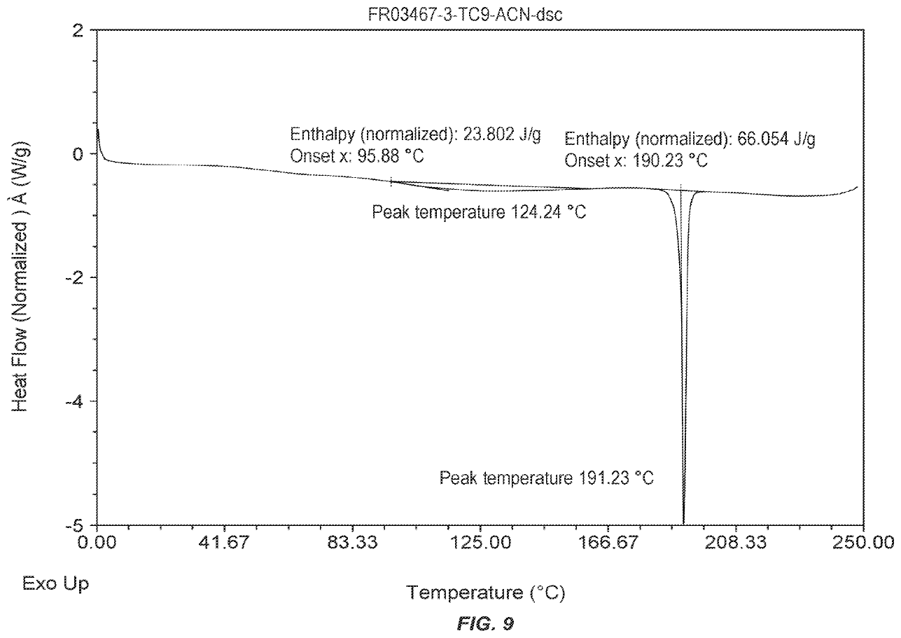

A representative differential scanning calorimetry (DSC) thermogram of Form B is presented in FIG. 9. In one embodiment, provided herein is a solid form comprising a free base of Compound 1, which exhibits, as characterized by DSC, a thermal event with an onset temperature of about 96° C. and a second thermal event with an onset temperature of about 190° C. In one embodiment, the first thermal event also has a peak temperature of about 124° C., and the second thermal event also has a peak temperature of about 191° C. In one embodiment, without being limited by any particular theory, the second thermal event corresponds to melting of Form B. In one embodiment, provided herein is a solid form comprising a free base of Compound 1, characterized by a DSC thermogram substantially as shown in FIG. 9.

In one embodiment, Form B of a free base of Compound 1 is prepared as discussed herein in section 5.2.

In one embodiment, provided herein is a solid form comprising Form B of a free base of Compound 1 and the amorphous form of a free base of Compound 1 provided herein. In one embodiment, provided herein is a solid form comprising Form B of a free base Compound 1 and one or more other crystalline forms of a free base of Compound 1 provided herein.

In another embodiment, Form B is substantially pure. In another embodiment, Form B is substantially physically pure. In certain embodiments, the substantially physically pure Form B is substantially free of other solid forms, e.g., amorphous solid. In certain embodiments, the purity of the substantially physically pure Form B is no less than about 95%, no less than about 96%, no less than about 97%, no less than about 98%, no less than about 98.5%, no less than about 99%, no less than about 99.5%, or no less than about 99.8%.

In certain embodiments, the composition comprising Form B is substantially physically pure. In certain embodiments, the composition comprising Form B is substantially free from other solid forms, for example, as containing no detectable amount of another solid form (e.g., another polymorph such as Form A or Form C). In certain embodiments, the composition comprising Form B contains no detectable amount of another solid form (e.g., another polymorph such as Form A or Form C), as determined by observing no detectable differences in an XRPD and/or DSC pattern between a single Form B crystal and the crystalline composition of Compound 1 ((5R,6S)-10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide).

### Form C

In certain embodiments, provided herein is Form C.

In one embodiment, Form C is a solid form of Compound 1. In one embodiment, Form C is a solid form of a free base of Compound 1. In another embodiment, Form C is crystalline. In one embodiment, Form C is a solvate solid form of Compound 1. In one embodiment, Form C is a solvate form of a free base of Compound 1.

A representative XRPD pattern of Form C is provided in FIG. 11.

In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, or all of the peaks located at approximately the following positions: 8.3, 9.1, 10.5, 11.5, 11.7, 12.6, 13.9, 14.6, 14.9, 15.4, 16.6, 17.4, 18.1, 18.4, 19.2, 19.7, 20.2, 20.7, 21.1, 21.4, 21.9, 22.2, 23.4, 23.6, 24.1, 24.8, 25.0, 25.4, 25.9, 26.9, 27.4, 27.9, 28.4, 28.7, 29.4, 29.9, 31.0, 31.2, 31.7, 32.0, 32.7, 33.3, 34.0, 34.3, 35.0, 35.3, 36.1, 36.9, 37.2, 38.0, 38.6, and 39.2° 2θ. In one embodiment, provided herein is a solid form comprising a free base of Compound 1, characterized by 1, 2, 3, 4, 5, 6, 7, 8, 9, 10, 11, 12, 13, 14, 15, 16, 17, 18, 19, 20, 21, 22, or all of the peaks located at approximately the following positions: 8.29, 9.15, 10.52, 11.48, 11.74, 12.60, 13.87, 14.61, 14.91, 15.41, 16.65, 17.43, 18.13, 18.40, 19.18, 19.68, 20.24, 20.73, 21.14, 21.37, 21.87, 22.26, 23.43, 23.61, 24.10, 24.76, 25.04, 25.38, 25.95, 26.88, 27.46, 27.94, 28.38, 28.70, 29.46, 29.92, 30.96, 31.29, 31.68, 31.98, 32.74, 33.30, 34.03, 34.32, 35.05, 35.32, 36.10, 36.86, 37.17, 37.97, 38.60, and 39.25° 2θ. In one embodiment, the solid form is characterized by 3 of the peaks. In one embodiment, the solid form is characterized by 5 of the peaks. In one embodiment, the solid form is characterized by 7 of the peaks. In one embodiment, the solid form is characterized by 9 of the peaks. In one embodiment, the solid form is characterized by 11 of the peaks. In one embodiment, the solid form is characterized by 15 of the peaks. In one embodiment, the solid form is characterized by 25 of the peaks. In one embodiment, the solid form is characterized by all of the peaks.

In one embodiment, provided herein is a solid form comprising a free base of Compound 1, characterized by an XRPD pattern comprising peaks at approximately 8.3, 12.6, and 16.6° 2θ. In one embodiment, the XRPD pattern further comprises peaks at approximately 17.4, 18.1, and 18.4° 2θ. In one embodiment, the XRPD pattern further comprises peaks at approximately 20.7, 23.4, and 23.6° 2θ. In one embodiment, the XRPD pattern further comprises peaks at approximately 25.4, 26.9, 27.9, and 29.9° 2θ. In one embodiment, the XRPD pattern comprises peaks at approximately 16.6, 17.4, 18.1, and 18.4° 2θ. In one embodiment, the XRPD pattern comprises peaks at approximately 20.7, 21.1, 21.4, and 21.9° 2θ. In one embodiment, the XRPD pattern comprises peaks at approximately 23.4, 23.6, and 24.1° 2θ. In one embodiment, the XRPD pattern comprises peaks at approximately 24.8, 25.0, 25.4, 25.9, and 26.9° 2θ. In one embodiment, the XRPD pattern comprises peaks at approximately 27.9 and 28.4° 2θ. In one embodiment, the XRPD pattern comprises peaks at approximately 29.9, 31.0° 2θ. In one embodiment, the XRPD pattern comprises peaks at approximately 8.3, 9.1, 10.5, 11.5, 11.7, 12.6, 16.6, 17.4, 18.1, 18.4, 20.7, 21.1, 21.4, 21.9, 23.4, 23.6, 24.1, 24.8, 25.0, 25.4, 25.9, 26.9, 27.9, 28.4, 29.9, 31.0, 31.3, 31.7, and 32.7° 2θ.

In one embodiment, provided herein is a solid form comprising a free base of Compound 1, characterized by an XRPD pattern comprising peaks at approximately 8.3, 12.6, and 16.6° 2θ±0.04° 2θ. In one embodiment, the XRPD pattern further comprises peaks at approximately 17.4, 18.1, and 18.4° 2θ±0.04° 2θ. In one embodiment, the XRPD pattern further comprises peaks at approximately 20.7, 23.4, and 23.6° 2θ±0.04° 2θ. In one embodiment, the XRPD pattern further comprises peaks at approximately 25.4, 26.9, 27.9, and 29.9° 2θ±0.04° 2θ. In one embodiment, the XRPD pattern comprises peaks at approximately 16.65, 17.43, 18.13, and 18.40° 2θ±0.04° 2θ. In one embodiment, the XRPD pattern comprises peaks at approximately 20.7, 21.14, 21.37, and 21.87° 2θ±0.04° 2θ. In one embodiment, the XRPD pattern comprises peaks at approximately 23.43, 23.61, and 24.10° 2θ±0.04° 2θ. In one embodiment, the XRPD pattern comprises peaks at approximately 24.76, 25.04, 25.38, 25.95, and 26.88° 2θ±0.04° 2θ. In one embodiment, the XRPD pattern comprises peaks at approximately 27.94 and 28.38° 2θ±0.04° 2θ. In one embodiment, the XRPD pattern comprises peaks at approximately 29.92, 30.96° 2θ±0.04° 2θ. In one embodiment, the XRPD pattern comprises peaks at approximately 8.3, 9.1, 10.5, 11.5, 11.7, 12.6, 16.6, 17.4, 18.1, 18.4, 20.7, 21.1, 21.4, 21.9, 23.4, 23.6, 24.1, 24.8, 25.0, 25.4, 25.9, 26.9, 27.9, 28.4, 29.9, 31.0, 31.3, 31.7, and 32.7° 2θ±0.04° 2θ.

In one embodiment, provided herein is a solid form comprising a free base of Compound 1, characterized by an XRPD pattern comprising peaks at approximately 8.3, 12.6, and 16.6° 2θ±0.02° 2θ. In one embodiment, the XRPD pattern further comprises peaks at approximately 17.4, 18.1, and 18.4° 2θ±0.02° 2θ. In one embodiment, the XRPD pattern further comprises peaks at approximately 20.7, 23.4, and 23.6° 2θ±0.02° 2θ. In one embodiment, the XRPD pattern further comprises peaks at approximately 25.4, 26.9, 27.9, and 29.9° 2θ±0.02° 2θ. In one embodiment, the XRPD pattern comprises peaks at approximately 16.65, 17.43, 18.13, and 18.40° 2θ±0.02° 2θ. In one embodiment, the XRPD pattern comprises peaks at approximately 20.7, 21.14, 21.37, and 21.87° 2θ±0.02° 2θ. In one embodiment, the XRPD pattern comprises peaks at approximately 23.43, 23.61, and 24.10° 2θ±0.02° 2θ. In one embodiment, the XRPD pattern comprises peaks at approximately 24.76, 25.04, 25.38, 25.95, and 26.88° 2θ±0.02° 2θ. In one embodiment, the XRPD pattern comprises peaks at approximately 27.94 and 28.38° 2θ±0.02° 2θ. In one embodiment, the XRPD pattern comprises peaks at approximately 29.92, 30.96° 2θ±0.02° 2θ. In one embodiment, the XRPD pattern comprises peaks at approximately 8.3, 9.1, 10.5, 11.5, 11.7, 12.6, 16.6, 17.4, 18.1, 18.4, 20.7, 21.1, 21.4, 21.9, 23.4, 23.6, 24.1, 24.8, 25.0, 25.4, 25.9, 26.9, 27.9, 28.4, 29.9, 31.0, 31.3, 31.7, and 32.7° 2θ±0.02° 2θ.

In one embodiment, provided herein is a solid form comprising a free base of Compound 1, characterized by an XRPD pattern substantially as shown in FIG. 11.

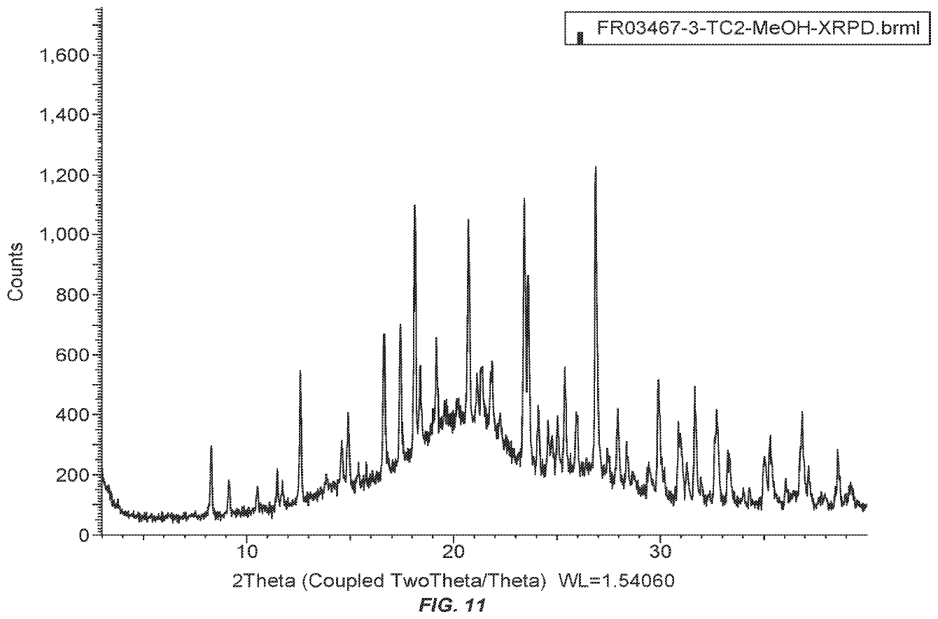

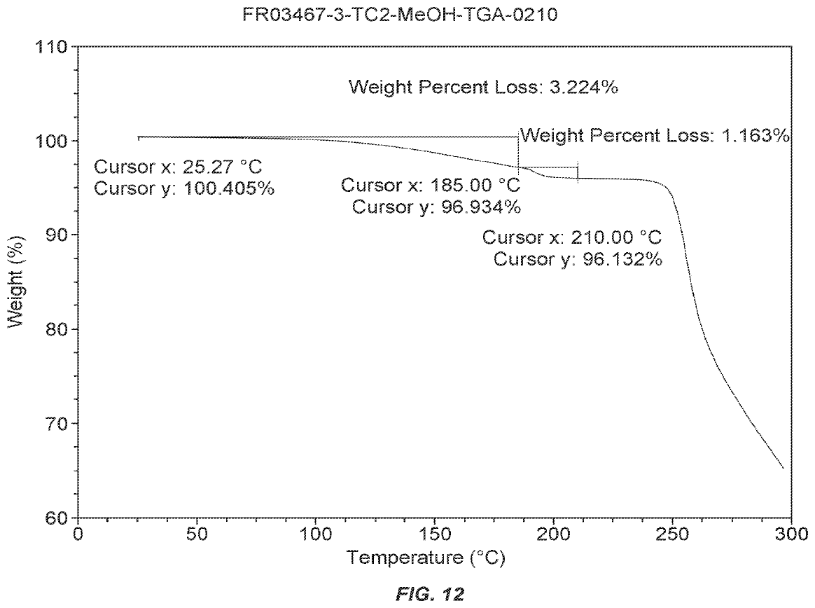

A representative thermal gravimetric analysis (TGA) thermogram of Form C is provided in FIG. 12. In one embodiment, provided herein is a solid form comprising a free base of Compound 1, which exhibits a thermal gravimetric analysis plot comprising two mass losses of about 3.2% and about 1.2% when heated from about 30° C. to about 260° C. In one embodiment, the solid form comprising a free base of Compound 1 exhibits about 3.2% mass loss when heated from about room temperature to about 185° C. and 1.2% mass loss when heated from about 185° C. to about 210° C. In one embodiment, without being limited by any particular theory, the weight loss corresponds to the loss of methanol. In one embodiment, the solid form comprising a free base of Compound 1 is a solvate. In one embodiment, the solid Form C comprising a free base of Compound 1 is a methanol solvate. In one embodiment, provided herein is a solid form comprising a free base of Compound 1, characterized by a TGA thermogram substantially as shown in FIG. 12.

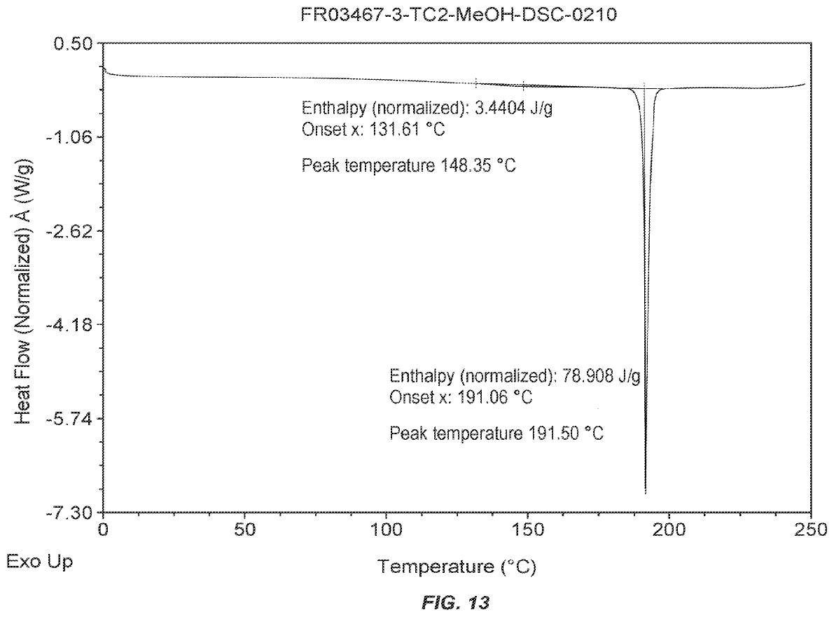

A representative differential scanning calorimetry (DSC) thermogram of Form C is presented in FIG. 13. In one embodiment, provided herein is a solid form comprising a free base of Compound 1, which exhibits, as characterized by DSC, a thermal event with an onset temperature of about 132° C. and a second thermal event with an onset temperature of about 191° C. In one embodiment, the first thermal event also has a peak temperature of about 148° C., and the second thermal event also has a peak temperature of about 191.5° C. In one embodiment, without being limited by any particular theory, the second thermal event corresponds to melting of Form C. In one embodiment, provided herein is a solid form comprising a free base of Compound 1, characterized by a DSC thermogram substantially as shown in FIG. 13.

In one embodiment, Form C of a free base of Compound 1 is prepared as discussed herein in section 5.2.

In one embodiment, provided herein is a solid form comprising Form C of a free base of Compound 1 and the amorphous form of a free base of Compound 1 provided herein. In one embodiment, provided herein is a solid form comprising Form C of a free base Compound 1 and one or more other crystalline forms of a free base of Compound 1 provided herein.

In another embodiment, Form C is substantially pure. In another embodiment, Form C is substantially physically pure. In certain embodiments, the substantially physically pure Form C is substantially free of other solid forms, e.g., amorphous solid. In certain embodiments, the purity of the substantially physically pure Form C is no less than about 95%, no less than about 96%, no less than about 97%, no less than about 98%, no less than about 98.5%, no less than about 99%, no less than about 99.5%, or no less than about 99.8%.

In certain embodiments, the composition comprising Form C is substantially physically pure. In certain embodiments, the composition comprising Form C is substantially free from other solid forms, for example, as containing no detectable amount of another solid form (e.g., another polymorph such as Form A or Form B). In certain embodiments, the composition comprising Form C contains no detectable amount of another solid form (e.g., another polymorph such as Form A or Form B), as determined by observing no detectable differences in an XRPD and/or DSC pattern between a single Form C crystal and the crystalline composition of Compound 1 ((5R,6S)-10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide).

### Amorphous Form

In certain embodiments, provided herein is an amorphous solid of Compound 1.

In one embodiment, provided herein is a solid form of a free base of Compound 1.

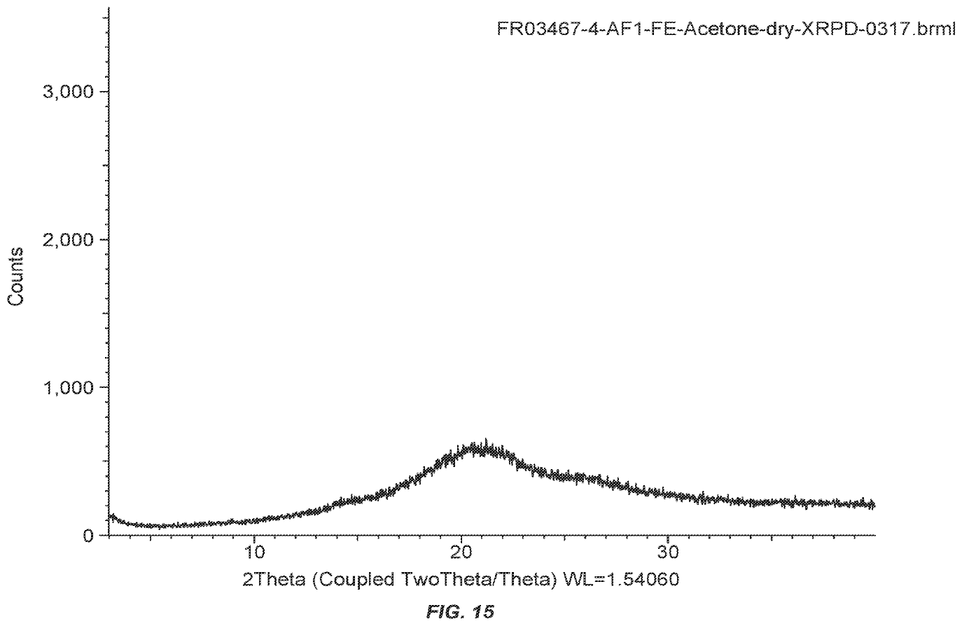

In one embodiment, the amorphous solid comprising a free base of Compound 1, is characterized by an XRPD pattern substantially as shown in FIG. 15.

In one embodiment, provided herein is an amorphous solid of Compound 1 having a DSC thermogram as depicted in comprising a glass transition temperature of 106.6° C. when heated from approximately 25° C. to approximately 300° C.

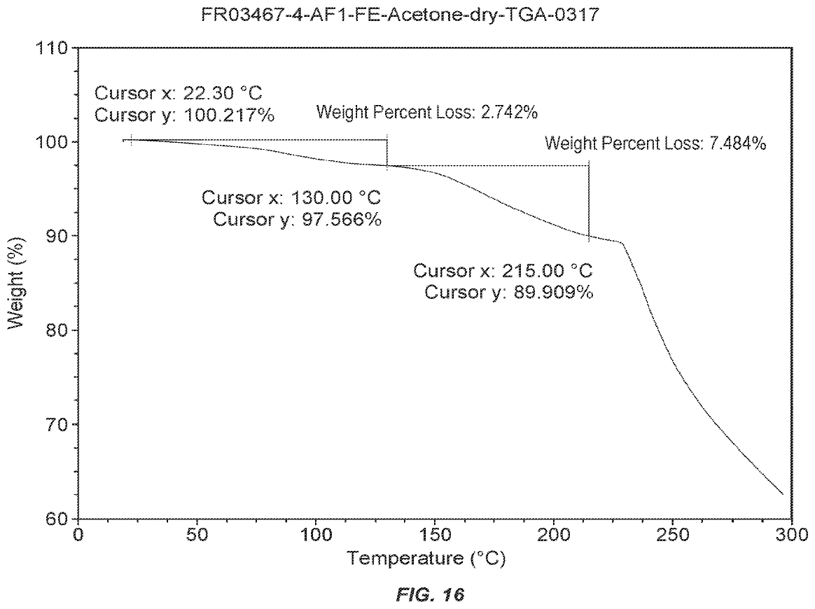

A representative thermal gravimetric analysis (TGA) thermogram of amorphous form is provided in FIG. 16. In one embodiment, provided herein is an amorphous solid form comprising a free base of Compound 1, which exhibits a thermal gravimetric analysis plot comprising two mass losses of about 2.7% and about 7.5% when heated from about 30° C. to about 260° C. In one embodiment, the amorphous solid form comprising a free base of Compound 1 exhibits about 2.7% mass loss when heated from about room temperature to about 130° C. and 7.5% mass loss when heated from about 130° C. to about 215 CC. In one embodiment, provided herein is an amorphous solid form comprising a free base of Compound 1, characterized by a TGA thermogram substantially as shown in FIG. 16.

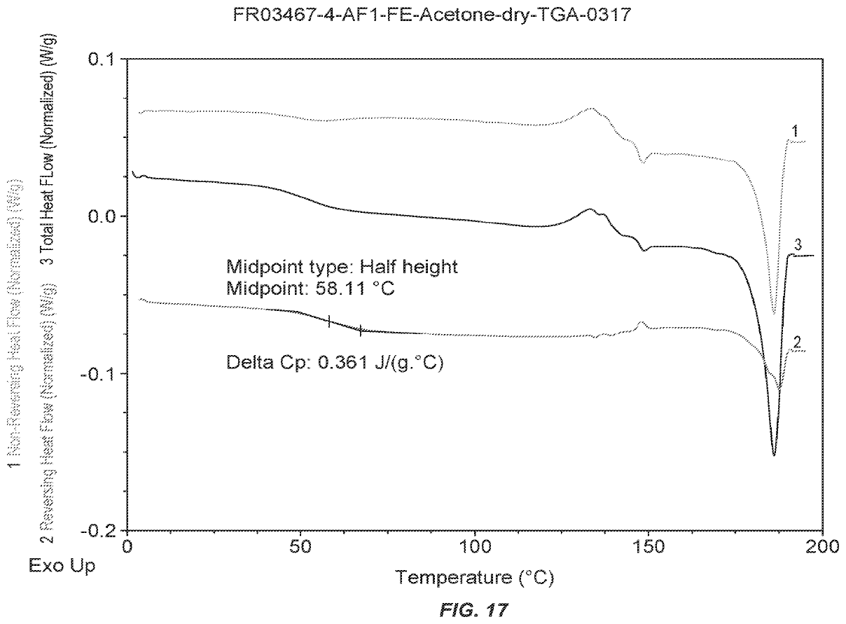

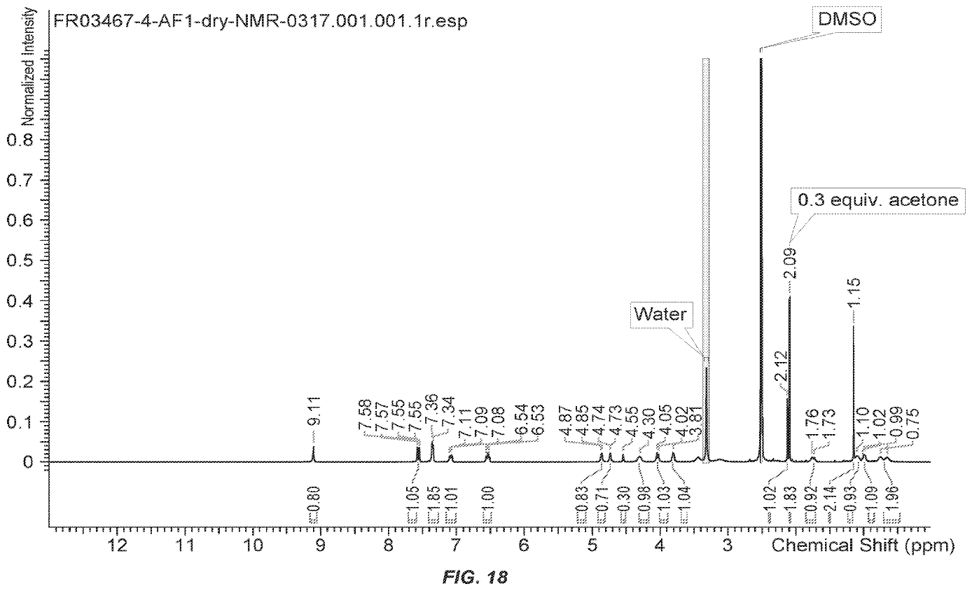

A representative differential scanning calorimetry (DSC) thermogram of an amorphous solid of Compound 1 is presented in FIG. 17. In one embodiment, provided herein is an amorphous solid form comprising a free base of Compound 1, which exhibits, as characterized by DSC, a glass transition temperature of 58° C. when heated from approximately 25° C. to approximately 200° C. In one embodiment, provided herein is an amorphous solid form comprising a free base of Compound 1, characterized by a DSC thermogram substantially as shown in FIG. 17.

### 6.2 Methods of Preparation

Also provided are methods for preparing the solid forms comprising a free base of Compound 1 in an amorphous form, or crystalline form.

In certain embodiments, solid forms provided herein are obtained by equilibration experiments, evaporation experiments, cooling recrystallization experiments, vapour diffusion experiments, and anti-solvent recrystallization experiments.

In one embodiment, the methods comprise the step of contacting Compound 1 with a solvent, in which the particulates of the Compound 1 in an amorphous form, or crystalline form (e.g., Form A, B, or C) of a free base of Compound 1, may be formed from a solution or converted from one solid form to another. The process may further comprise an isolation step, in which the compounds may be isolated by a conventional method, such as filtration and centrifugation, followed by washing with a solvent and then drying (e.g., vacuum oven drying, air drying, or desiccator drying).

Suitable solvents for use in preparing the compounds in an amorphous form, or crystalline form, include but are not limited to, hydrocarbons, including petroleum ether, pentane, hexane(s), heptane, octane, isooctane, cyclopentane, cyclohexane, methylcyclohexane, benzene, toluene, xylene, tetralin, and cumene; chlorinated hydrocarbons, including dichloromethane (DCM), 1,2-dichloroethane, 1, 1-dichloroethene, 1,2-dichloroethene, chloroform, trichloroethane, trichloroethene, carbon tetrachloride, chlorobenzene, and trifluoromethylbenzene; alcohols, including methanol, ethanol, isopropanol (IPA), 1-propanol, 1-butanol, 2-butanol, t-butanol, 3-methyl-1-butanol, 1-pentanol, 2-methoxyethanol, 2-ethoxyethanol, and ethyleneglycol; ethers, including diethyl ether, diisopropyl ether, methyl t-butyl ether (MTBE), diphenyl ether, 1,2-dimethoxyethane, bi(2-methoxyethyl)ether, 1,1-dimethoxymethane, 2,2-dimethoxypropane, and anisole; ketones, including acetone, butanone, methyl ethyl ketone (MEK), methyl isopropyl ketone, methyl butyl ketone, and methyl isobutyl ketone (MIBK); esters, including methyl acetate, ethyl formate, ethyl acetate, propyl acetate, isopropyl acetate, isobutyl acetate, and butyl acetate; carbonates, including ethylene carbonate and propylene carbonate; amides, including formamide, N,N-dimethylformamide (DMF), and N,N-dimethylacetamide; nitriles, including acetonitrile (ACN); sulfoxides, such as dimethyl sulfoxide (DMSO); sulfones, such sulfolane; nitro compounds, such as nitromethane and nitrobenzene; heterocycles, such as N-methyl pyrrolindone, 2-methyl tetrahydrofuran, tetrahydrofuran (THF), dioxane, and pyridine; carboxylic acids, such as acetic acid, trichloroacetic acid, and trifluoroacetic acid; phosphoramides, such as hexamethylphosphoramide; carbon sulfide; water; and mixtures thereof.

In one embodiment, Form A of Compound 1 is prepared by equilibration in a solvent at about room temperature. In one embodiment, Form A of Compound 1 can be prepared by equilibration in a solvent at about 50° C. In one embodiment, Form A of Compound 1 can be prepared by equilibration under a temperature cycle. In one embodiment, the temperature cycle is between about 5 to about 50° C. In one embodiment the solvent is selected from isopropanol, 2-methyltetrahydrofuran, toluene, heptane, methyl tert-butyl ether, methyl isobutyl ketone, ethyl acetate, ethanol, water or a mixture thereof. In one embodiment, the solvent is selected from ispropanol/water, methyl tert-butyl ether/methyl isobutyl ketone, 2-methyltetrahydrofuran/heptane, ethylacetate/heptane, or ethanol/heptane.

In one embodiment, Form A of Compound 1 is prepared by addition of an antisolvent. In one embodiment, Form A is formed by: 1) dissolving or slurring Compound 1 in a minimal amount of a solvent, 2) filtering the solution or suspension; and 3) adding an anti-solvent into the clear solution. In one embodiment, the solvent is methanol, water, 2-methyltetrahydrofuran, heptane, ethyl acetate, ethanol or a combination thereof. In one embodiment, the solvent is methanol/water.

In one embodiment, Form A of Compound 1 is prepared by vapor diffusion. In one embodiment, the solvent is isopropanol, hexane, or a combination thereof. In one embodiment, the solvent is isopropanol/hexane.

In one embodiment, Form A is prepared by interconversion of Form B. In one embodiment Form A is prepared by interconversion of Form C. In one embodiment, Form A is prepared from Form B upon heating Form B at about 170° C. In one embodiment, Form A is prepared from Form C upon heating Form C at about 185° C.

In one embodiment, Form A can be prepared from the amorphous form by equilibration in a solvent. In one embodiment, the solvent is isopropanol, etahnol, toluene, heptane, ethyl acetate, methyl tert-butyl ether or a combination thereof.

In one embodiment, Form B of Compound 1 is prepared by equilibration in a solvent at about room temperature. In one embodiment, the solvent is acetonitrile. In one embodiment, Form B is prepared by equilibration of Form A in acetonitrile. In one embodiment, Form B is prepared by equilibration of amorphous form of Compound 1 in acetonitrile.

In one embodiment, Form C of Compound 1 is prepared by equilibration in a solvent at about room temperature. In one embodiment, the solvent is methanol. In one embodiment, Form C is prepared by equilibration of Form A in methanol. In one embodiment, Form C is prepared by equilibration of amorphous form of Compound 1 in methanol.

In one embodiment, the amorphous form of a free base of Compound 1 is prepared by evaporation experiments, vapor diffusion experiments, and anti-solvent recrystallization experiments. In one embodiment, the amorphous form of Compound 1 is prepared from a solvent selected from isopropanol, heptane, DMSO, water, ethyl acetate, hexane, ethanol, isopropanol, acetone, 2-methyltetrahydrofuran, methyl isobutyl ketone, ethyl acetate, methanol, ethanol, tetrahydrofuran, water or a mixture thereof.

In one embodiment, the amorphous form of a free base of Compound 1 is prepared by evaporation experiments in a solvent selected from ethanol, isopropanol, acetone, 2-methyltetrahydrofuran, methyl isobutyl ketone, ethyl acetate, methanol, ethanol, tetrahydrofuran, water or a mixture thereof.

In one embodiment, the amorphous form of a free base of Compound 1 is prepared by vapour diffusion experiments in ethyl acetate, hexane, or a mixture thereof. In one embodiment, the amorphous form of a free base of Compound 1 is prepared by vapor diffusion experiments in ethyl acetate/hexane.

In one embodiment, the amorphous form of Compound 1 is prepared by addition of an anti-solvent. In one embodiment, the amorphous form is formed by: 1) dissolving or slurring Compound 1 in a minimal amount of a solvent, 2) filtering the solution or suspension; and 3) adding an anti-solvent into the clear solution. In one embodiment, the solvent is isopropanol, heptane, DMSO, water, or a mixture thereof. In one embodiment, the solvent is isopropanol/heptane, or DMSO/water.

In one embodiment, the amorphous form of Compound 1 is prepared by vapor diffusion. In one embodiment, the solvent is ethyl acetate, hexane, or a combination thereof. In one embodiment, the solvent is ethyl acetate/hexane.

Other forming methods may also be applicable for preparing the solid forms of Compound 1 in an amorphous form, or crystalline forms (e.g., Form A, Form B, or Form C), including spray drying, roller drying, lyophilization, and melt crystallization.

### 6.3 Methods of Use

In one embodiment, provided herein is a method of treating a disease or disorder in which the RAS pathway is upregulated such as cancer, RASopathies and laminopathies, which comprises administering to a patient a solid form of Compound 1 provided herein. In one embodiment, provided herein is a method of treating cancer. In one embodiment, provided herein is a solid form of Compound 1 provided herein for use in a method of treating a disease or disorder in which the RAS pathway is upregulated such as cancer, RASopathies and laminopathies, wherein the method comprises administering said solid form to a patient. In one embodiment, provided herein is a solid form of Compound 1 provided herein a solid form of Compound 1 provided herein for use in a method of treating cancer.

In one embodiment, provided herein is a method of preventing a disease or disorder in which the RAS pathway is upregulated such as cancer, RASopathies and laminopathies, which comprises administering to a patient a solid form of Compound 1 provided herein. In one embodiment, provided herein is a method of preventing cancer. In one embodiment, provided herein is a solid form of Compound 1 provided herein for use in a method of preventing a disease or disorder in which the RAS pathway is upregulated such as cancer, RASopathies and laminopathies, wherein the method comprises administering said compound to a patient. In one embodiment, provided herein is a solid form of Compound 1 provided herein for use in a method of preventing cancer.

In one embodiment, provided herein is a method of managing a disease or disorder in which the RAS pathway is upregulated such as cancer, RASopathies and laminopathies, which comprises administering to a patient a solid form of Compound 1 provided herein. In one embodiment, provided herein is a method of managing cancer. In one embodiment, provided herein is a solid form of Compound 1 provided herein for use in a method of managing a disease or disorder in which the RAS pathway is upregulated such as cancer, RASopathies and laminopathies, wherein the method comprises administering said compound to a patient. In one embodiment, provided herein is a solid form of Compound 1 provided herein for use in a method of managing cancer.

In some embodiments, RASopathies include, but are not limited to, neurofibromatosis 1 (NF1), neurofibromatosis 2 (NF2), Noonan Syndrome, Costello Syndrome, Cardiofaciocutaneous Syndrome, Legius Syndrome, Capillary Arteriovenous Malformation Syndrome, Hereditary gingival fibromatosis, or SYNGAP1 syndrome. In certain embodiments, NF1 is associated with cutaneous and/or plexiform neurofibromas.

In some embodiments laminopathies include, but are not limited to, the Atypical Werner syndrome, Buschke-Ollendorff syndrome, Cardiomyopathy, Charcot-Marie-Tooth disease, Emery-Dreifuss muscular dystrophies, Greenberg dysplasia, Hutchinson-Gilford progeria syndrome (HGPS), Leukodystrophy, demyelinating, adult-onset, autosomal dominant (ADLD), Limb-girdle muscular dystrophy type 1B (LGMD1B), Lipoatrophy with diabetes, hepatic steatosis, hypertrophic cardiomyopathy, and leukomelanodermic papules (LDHCP), Mandibuloacral dysplasia, Pelger-Huet anomaly (PHA), or Restrictive dermopathy.

In one particular embodiment of the methods described herein, the disease or disorder is a Neurofibromatosis (NF) related disorder. In some embodiments, the neurofibromatosis is neurofibromatosis 1 (NF1), neurofibromatosis 2 (NF2), or schwannomatosis. In some such embodiments, the disease or disorder is cancer. In one particular embodiment of the methods described herein, the disease or disorder is a Neurofibromatosis Type 1 (NF1)-associated cancer. In one particular embodiment, provided herein is a method of treating NF1 in a subject having NF1. In some embodiments, the methods provided herein comprise treating cutaneous and/or plexiform neurofibromas in NF1 in a subject having NF1. In one embodiment of the methods described herein, the disease or disorder is a Neurofibromatosis Type 2 (NF2)-associated cancer. In one embodiment of the methods described herein, the disease or disorder is schwannomatosis. In one embodiment of the methods described herein, the disease or disorder is a schwannomatosis-associated cancer.

In other aspects, provided herein is a method for the treatment, prevention or prophylaxis of cancer comprising administering to a subject in need thereof an effective amount of one or more solid forms of Compound 1 described herein or a pharmaceutical composition that includes one or more solid forms of Compound 1 described herein. In certain embodiments, the cancer may be selected from brain cancer, breast cancer, lung cancer, ovarian cancer, pancreatic cancer, stomach cancer, prostate cancer, renal cancer, colorectal cancer, melanoma or leukemia. In further or additional embodiments, the cancer is brain cancer or achenocortical carcinoma. In further or additional embodiments, the cancer is breast cancer. In further or additional embodiments, the cancer is ovarian cancer. In further or additional embodiments, the cancer is pancreatic cancer. In further or additional embodiments, the cancer is stomach cancer. In further or additional embodiments, the cancer is prostate cancer. In further or additional embodiments, the cancer is renal cancer. In further or additional embodiments, the cancer is colorectal cancer. In further or additional embodiments, the cancer is myeloid leukemia. In further or additional embodiments, the cancer is glioblastoma. In further or additional embodiments, the cancer is follicular lymphona. In further or additional embodiments, the cancer is pre-B acute leukemia. In further or additional embodiments, the cancer is chronic lymphocytic B-leukemia. In further or additional embodiments, the cancer is mesothelioma. In further or additional embodiments, the cancer is small cell lung cancer. In a particular embodiment, the cancer is low grade serious ovarian cancer.

In some embodiments, provided herein is a method of inhibiting proliferation of a cell having a RAS mutation, comprising administering one or more solid forms of Compound 1 described herein. In some embodiments, the cancer is associated with a RAS mutation. In certain embodiments, provided herein is a method of inducing apoptosis in a cell having a RAS mutation, comprising administering one or more solid forms of Compound 1 described herein. In some embodiments, provided herein is a method of inhibiting proliferation of a cell having a KRAS mutation, comprising administering one or more solid forms of Compound 1 described herein. In some embodiments, the cancer is associated with a KRAS mutation. In some embodiments, provided herein is a method of inducing apoptosis in a cell in a cell having a KRAS mutation, comprising administering one or more solid forms of Compound 1 described herein. In some embodiments, provided herein is a method of inhibiting proliferation of a cell having a NRAS mutation, comprising administering one or more solid forms of Compound 1 described herein. In some embodiments, the cancer is associated with a NRAS mutation. In some embodiments, provided herein is a method of inducing apoptosis in a cell having a RAS mutation, comprising administering one or more solid forms of Compound 1 described herein. In some embodiments, the KRAS mutation is at codons 12, 13, 59, 61 and/or 146. In some embodiments, the mutant form of the KRAS protein has one or more amino acid substitutions selected from the group consisting of G12C, G12S, G12R, G12F, G12L, G12N, G12A, G12D, G12V, G13C, G13S, G13D, G13V, G13P, S17G, P34S, A59E, A59G, A59T, Q61K, Q61L, Q61R, and Q61H. In some embodiments, the mutant form of the KRAS protein has one or more amino acid substitutions selected from the group consisting of G12C, G12R, G12S, G12A, G12D, G12V, G13C, G13R, G13S, G13A, G13D, G13V, A59E, A59G, A59T, Q61K, Q61L, Q61R, Q61H, K117N, K117R, K117E, A146P, A146T and A146V.

In certain embodiments, the RAS mutation is a HRAS or MRAS mutation. In one embodiment, the mutant form of the HRAS protein has one or more amino acid substitutions selected from the group consisting of G12C, G12D, G12F, G12N, G12S, G12V, G13C, G13D, G13E, G13R, G13S, G13V, Q61H, Q61K, Q61L, and Q61R.

In one embodiment, the mutant form of the MRAS protein has one or more amino acid substitutions selected from G23V and T68I.

In some embodiments, provided herein is a method of treating cancer in a subject having a RAF mutation, comprising administering one or more solid forms of Compound 1 described herein. In some embodiments, provided herein is a method of inhibiting proliferation of a cell having a RAF mutation, comprising administering one or more solid forms of Compound 1 described herein. In some embodiments, the cancer is associated with a RAF mutation. In some embodiments, the BRAF mutation is a K601E mutation. In some embodiments, the BRAF mutation is a V600E mutation.

In some embodiments, provided herein is a method of treating cancer in a subject having a NF1 mutation, comprising administering one or more solid forms of Compound 1 described herein.

In certain embodiments, the cancer is resistant to treatment of one or more MEK protein kinase inhibitors. In other embodiments, the cancer is resistant to treatment of one or more RAF protein kinase inhibitors. In some embodiments, the cancer is resistant to BRAF/MEK inhibition. In some such embodiments, the cancer is an advanced solid tumor. In some embodiments, the cancer is associated with a RAS, RAF, and/or NF1 mutation. In some embodiments, the BRAF mutation is a K601E mutation. In some embodiments, the BRAF mutation is a V600E mutation. In still further embodiments, the resistance is acquired resistance. In other embodiments, the resistance is de novo resistance. In further or additional embodiments, the cancer is resistant to an anticancer agent.

In some embodiments, a solid form of Compound 1 provided herein can be administered once daily (QD or qd), or divided into multiple daily. In addition, the administration can be continuous (i.e., daily for consecutive days or every day), intermittent, e.g., in cycles (i.e., including days, weeks, or months of rest without drug). As used herein, the term “daily” is intended to mean that a therapeutic compound is administered once or more than once each day, for example, for a period of time.

In some embodiments, administration of a compound or pharmaceutical composition disclosed herein may occur in an amount of between about 0.001 mg/kg of body weight to about 100 mg/kg of body weight per day (administered in single or divided doses), more preferably at least about 0.1 mg/kg of body weight per day. A particular therapeutic dosage can include, e.g., from about 0.01 mg to about 7000 mg of compound, and preferably includes, e.g., from about 0.05 mg to about 2500 mg. The quantity of active compound in a unit dose of preparation may be varied or adjusted from about 0.1 mg to 1000 mg, preferably from about 1 mg to 300 mg, more preferably 10 mg to 200 mg, according to the particular application. In some instances, dosage levels below the lower limit of the aforesaid range may be more than adequate, while in other cases still larger doses may be employed without causing any harmful side effect, e.g. by dividing such larger doses into several small doses for administration throughout the day. The amount administered will vary depending on the particular IC₅₀ value of the compound used. In combinational applications in which the compound is not the sole therapy, it may be possible to administer lesser amounts of compound and still have therapeutic or prophylactic effect.

In one embodiment the compound or pharmaceutical composition disclosed herein is administered at a dosage amount of about 1 mg/day, 2 mg/day, 4 mg/day, 5 mg/day, 8 mg/day, 10 mg/day, 15 mg/day, 20 mg/day, 22 mg/day, 30 mg/day, 35 mg/day, 37 mg/day, 45 mg/day, 50 mg/day, 70 mg/day, 100 mg/day, 125 mg/day, 140 mg/day, 175 mg/day, 200 mg/day, 250 mg/day, 280 mg/day, 350 mg/day, 400 mg/day, 450 mg/day, 500 mg/day, 550 mg/day, 600 mg/day, 650 mg/day, 700 mg/day, 750 mg/day, 1000 mg/day or 1400 mg/day. In one embodiment the compound or pharmaceutical composition disclosed herein is administered at a dosage amount of about 2 mg/day, 4 mg/day, 8 mg/day, 15 mg/day, 22 mg/day, 30 mg/day, 37 mg/day, or 45 mg/day.

For clarity reasons, it is noted that, unless otherwise specified, the Compound 1 doses referred to herein refer to the amount of Compound 1 in its free base form. In case that for example a pharmaceutically acceptable salt of Compound 1 is used, the amounts given above will need to be adapted accordingly.

### 6.4 Pharmaceutical Compositions

Provided herein are pharmaceutical compositions. The pharmaceutical compositions provided herein contain therapeutically effective amounts of one or more of the solid forms of Compound 1 provided herein and optionally a pharmaceutically acceptable carrier, diluent or excipient.

The solid forms of the Compound 1 provided herein can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the solid forms provided herein described above are formulated into pharmaceutical compositions using techniques and procedures well known in the art (see, e.g., Ansel Introduction to Pharmaceutical Dosage Forms, Seventh Edition 1999). In one particular embodiment, the solid forms of the Compound 1 provided herein can be formulated into capsules. In some such embodiments, the the solid forms of the Compound 1 provided herein are formulated into 2 mg, 4 mg, or 15 mg capsule. In one particular embodiment, the solid forms of the Compound 1 provided herein are formulated into 15 mg capsule. In the compositions, effective concentrations of one or more solid forms of the Compound 1 provided herein or pharmaceutically acceptable salts thereof is (are) mixed with a suitable pharmaceutical carrier or vehicle. In certain embodiments, the concentrations of the solid forms of the Compound 1 provided herein in the compositions are effective for delivery of an amount, upon administration, that treats, prevents, or ameliorates one or more of the symptoms and/or progression of disorders in a subject, such as cancer.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, tissue distribution, inactivation, metabolism and excretion rates of the active compound, the physicochemical characteristics of the compound, the dosage schedule, and amount administered as well as other factors known to those of skill in the art. For example, the amount that is delivered is sufficient to ameliorate one or more of the symptoms of cancer or disease disclosed herein.

The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules.

Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof. Methods of preparing various pharmaceutical compositions with a specific amount of active compound are known, or will be apparent, to those skilled in this art. For examples, see Remington's Pharmaceutical Sciences, Mack Publishing Company, Ester, Pa., 18th Edition (1990).

In certain embodiments, the effective amount of the Compound 1 in the pharmaceutical composition may be at a level that will exercise the desired effect; for example, about 0.001 mg/kg of a subject's body weight to about 100 mg/kg of a subject's body weight in unit dosage for both oral and parenteral administration.

The dose of Compound 1 to be administered to a subject is rather widely variable and can be subject to the judgment of a health-care practitioner In any given case, the amount of Compound 1 administered will depend on such factors as the solubility of the active component, the formulation used and the route of administration. In one embodiment, application of a topical concentration provides intracellular exposures or concentrations of about 0.01-10 mM.

The administered dose can also be expressed in units other than mg/kg/day. For example, doses for parenteral administration can be expressed as mg/m²/day. One of ordinary skill in the art would readily know how to convert doses from mg/kg/day to mg/m²/day given either the height or weight of a subject or both. For example, a dose of 1 mg/kg/day for a 65 kg human is approximately equal to 38 mg/m²/day.

Compound 1 can be administered orally for reasons of convenience. In one embodiment, when administered orally, Compound 1 is administered with a meal and water. In one embodiment, when administered orally, Compound 1 is administered without food. In another embodiment, Compound 1 is dispersed in water or juice (e.g., apple juice or orange juice) and administered orally as a suspension.

Compound 1 can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

Compound 1 disclosed herein may be administered as a sole therapy. Compound 1 disclosed herein may also be administered in combination with another therapy or therapies.

Other therapies include, but are not limited to administration of other therapeutic agents, radiation therapy or both.

In the instances where the compound described herein is administered with other therapeutic agents, the compound described herein need not be administered in the same pharmaceutical composition as other therapeutic agents, and may because of different physical and chemical characteristics, be administered by a different route. For example, the compound or composition may be administered orally to generate and maintain good blood levels thereof, while the other therapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician. The particular choice of compound (and where appropriate, other therapeutic agent and/or radiation) will depend upon the diagnosis of the attending physicians and their judgment of the condition of the patient and the appropriate treatment protocol. Other therapeutic agents may include chemotherapeutic agents, such as anti-tumor substances. Such conjoint treatment may be achieved by way of the simultaneous, sequential or separate dosing of the individual components of treatment.

### 7. EXAMPLES

Certain embodiments of the invention are illustrated by the following non-limiting examples.

### Instrumental Methods

X-ray Powder Diffractometer (XRPD)

| | |
|---|---|
| Instrument | Bruker D8 Advance |
| Detector | LYNXEYE_XE_T(1D mode) |
| Open angle | 2.94° |
| Radiation | Cu/K-Alphal (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.12 second per step |
| Scan range | 3º to 40° |
| Sample rotation speed | 15 rpm |
| Sample holder | Monocrystalline silicon, flat surface |
| Sample treatment | Covered with Kapton (bring a halo background in the XRPD) |

Differential Scanning Calorimetry (DSC)

| | |
|---|---|
| Instrument | TA Discovery 2500 |
| Sample pan | Tzero pan and Tzero hermetic lid with a manually punched pin |
| | hole of about 0.7 mm in diameter |
| Temperature range | 0 to 250° C. |
| Heating rate | 10° C./min |
| Nitrogen flow | 50 mL/min |
| Sample mass | About 0.5-2 mg |

Modulated Differential Scanning Calorimetry (mDSC)

| | |
|---|---|
| Instrument | TA Instruments Discovery 2500 |
| Sample pan | Tzero pan and Tzero hermetic lid with a manually punched pin |
| | hole of about 0.7 mm in diameter |
| Temperature range | 0 to 200° C. |
| Modulation amplitude | ±0.32° C./min |
| Period of modulation | 60s |
| Heating rate | 2º C./min |
| Nitrogen flow | 50 mL/min |
| Sample mass | ~2-10 mg |

Thermal Gravimetric Analysis (TGA)

| | |
|---|---|
| Instrument | Discovery 5500 |
| Sample pan | Aluminum, open |
| Start temperature | Ambient condition (below 35° C.) |
| Final temperature | 300° C. or abort next segment if weight <80% (w/w) |
| | (The weight loss of the compound is no more than 20% (w/w)) |
| Heating rate | 10° C./min |
| Nitrogen flow | Balance 10 mL/min; sample chamber 25 mL/min |
| Sample mass | About 2-10 mg |

Dynamic Vapor Sorption (DVS)

| | |
|---|---|
| Instrument | Intrinsic Plus |
| Total gas flow | 200 sccm |
| Oven temperature | 25° C. |
| Solvent | Water |
| Method | Cycle: 40-0-95-0-40% RH, dm/dt 0.002%/min, stage step: |
| | 10% RH, minimum equilibration time 60 min/step, maximum |
| | equilibration time 360 min/step |
| Sample mass | About 5-30 mg |

Polarized Light Microscope (PLM)

| | |
|---|---|
| Instrument | Leica DM4 P |
| Method | Crossed polarizer, silicone oil added |

Scanning Electronic Microscope (SEM)

| | |
|---|---|
| Instrument | Phenom Prox SEM-EDS |
| Detector | BSD Full |
| Magnification (screen) | 200x-10,000x |

Nuclear Magnetic Resonance (NMR)

| | |
|---|---|
| Instrument | Bruker Avance-AV 400M |
| Frequency | 400 MHZ |
| Probe | 5 mm PABBO BB/19F-1H/D Z-GRD Z108618/0406 |
| Number of scan | 8 |
| Temperature | 297.6K |
| Relaxation delay | 1 second |

Fourier Transform Infrared Spectrum (FT-IR)

| | |
|---|---|
| Instrument | Thermo Nicolet iS5 |
| Number of scans | 32 |
| Mode | ATR |
| Resolution | 4 |
| Sample compartment | Main |
| Detector | DTGS KBr |
| Beamsplitter | KBr |
| Source | IR |
| Accessory | ID7 |
| Window | Diamond |
| Recommended range | 4000-525 cm⁻¹ |
| Gain: 1 | 1.0 |
| Optical velocity | 0.4747 |
| Aperture | 100 |

High Performance Liquid Chromatography (HPLC)

| Instrument | SHIMADZU LC-20AD | | |
|---|---|---|---|
| HPLC method | Wave length: 295 nm; Column: Zorbax SB-C18; | | |
| | Detector: SPD-M20A 230V; Column temperature: 40° C.; | | |
| | Flow rate: 1.2 mL/min | | |
| | Mobile phase A: 0.1% TFA in water | | |
| | Mobile phase B: ACN | | |
| | Diluent: ACN/water (1/1, v/v) | | |
| | Injection volume: 5uL | | |
| | Gradient: | | |
| | Time | Mobile Phase | Mobile Phase |
| | (min) | A (%) | B (%) |
| | 0.00 | 95 | 5 |
| | 0.01 | 95 | 5 |
| | 9.00 | 5 | 95 |
| | 13.00 | 5 | 95 |
| | 13.10 | 95 | 5 |
| | 17.00 | 95 | 5 |

Ion Chromatography (IC)

| | |
|---|---|
| Instrument | Metrohm 940 professional IC |
| Sample center | 889 IC |
| Detector | Conductivity detector |
| Eluent (anion) | 3.2 mmol/L Na₂CO₃ + 1.0 mmol/L NaHCO₃ |
| Eluent (cation) | 1.7 mmol/L HNO₃ + 0.7 mmol/L pyridinecarboxylic acid |
| Suppressor solutions | 2% H₃PO₄ |
| Column: | Anion A SUPP 5-150 or Cation Column C4-150 |
| Column temperature: | 30° C. |
| Flow rate: | 0.7 mL/min (anion) or 0.9 mL/min (cation) |
| Diluent: | ACN:water (1:1, v/v) |
| Injection volume: | 20 μL |

| Acronyms | Full name |
|---|---|
| XRPD | X-ray powder diffractometer |
| DSC | Differential scanning calorimetry |
| TGA | Thermal gravimetric analysis |
| PLM | Polarized light microscope |
| DVS | Dynamic vapor sorption |
| NMR | Nuclear magnetic resonance |
| HPLC | High performance liquid |
| | chromatography |
| IC | Ion chromatography |
| MeOH | Methanol |
| EtOH | Ethanol |
| IPA | Isopropanol |
| MIBK | Methyl iso-butyl ketone |
| EtOAc | Ethyl acetate |
| IPAc | Isopropyl acetate |
| MTBE | Methyl tert-butyl ether |
| ACN | Acetonitrile |
| THF | Tetrahydrofuran |
| 2-MeTHF | 2-Methyltetrahydrofuran |
| DMSO | Dimethyl sulfoxide |
| FaSSGF | Fasted state simulated gastric fluid |
| FaSSIF-v1 | Fasted state simulated intestinal |
| | fluid version 1 |
| FeSSIF-v1 | Fed state simulated intestinal |
| | fluid version 1 |
| SEM | Scanning electronic microscope |
| a.w. | Water activity |
| q.s. | Quantum satis |

FaSSGF, pH 1.6

| | | |
|---|---|---|
| Sodium taurocholate | 80 | μM |
| Lecithin | 20 | μM |
| Pepsin | 0.1 | mg/mL |
| NaCl | 34.2 | mM |
| HCl conc. (q.s.) | 1.6 | pH |
| Deionized water (q.s.) | 1 | L |
| PH | 1.6 | |

FaSSIF-v1, pH 6.5

| Sodium taurocholate | 3 | mM |
|---|---|---|
| Lecithin | 0.75 | mM |
| NaH₂PO₄ | 3.438 | g |
| NaCl | 6.186 | g |
| NaOH (q.s.) | 6.5 | pH |
| Deionized water (q.s.) | 1 | L |
| pH | 6.5 | |
| FeSSIF-v1, pH 5.0 | | |
| Sodium taurocholate | 15 | mM |
| Lecithin | 3.75 | mM |
| CH₃COOH | 8.65 | g |
| NaCl | 11.874 | g |
| NaOH | 4.04 | g |
| Deionized water (q.s.) | 1 | L |
| pH | 5.0 | |

### 7.1 Reference Synthesis of Compound 1

### Step 1: Preparation of 3,4,5-Trifluoro-1-nitrophenol

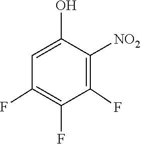 [SMILES: O=[N+]([O-])c1c(O)cc(F)c(F)c1F]

3,4,5-Trifluorophenol (14.81 g, 0.1 mol) was dissolved in glacial acetic acid (50 mL) and cooled to 4° C. while concentrated nitric acid (5 mL, 70%) was added dropwise over 15 min, during which time the color of the mixture becomes yellow. Upon complete addition of HNO₃, the reaction mixture was allowed to warm to room temperature and stirred for an additional 30 min. TLC analysis of an aliquot extracted into ethyl acetate indicates that a new non-polar spot was formed and the complete consumption of starting material. The mixture was then diluted with ethyl acetate (200 mL), transferred to separatory funnel, and washed copiously with water (3×100 mL). The organic layer was finally washed with brine, dried over anhydrous MgSO₄, and evaporated under reduced pressure to afford crude product as a yellowish oil. (17.3 g, 90%). This crude material was used directly in the subsequent reaction. ¹H NMR (400 MHz, CDCl₃): 6.84 (m, 1H, ArH), 10.28 (brs, 1H, OH).

### Step 2: Preparation of 3,4,5-Trifluoro-2-nitro-phenyl Ally Ether

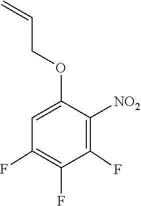 [SMILES: C=CCOc1cc(F)c(F)c(F)c1[N+](=O)[O-]]

To a solution of crude 3,4,5-trifluoro-2-nitrophenol (4.8 g, 25 mmol) in acetone (25 mL) was added K₂CO₃ (50 mmol) and allyl bromide (3.6 g, 30 mmol), and the mixture was heated to reflux for 2 h. TLC analysis of the reaction mixture (25% EtOAc:Hexanes) at this time reveals all starting material was consumed and the presence of a non-polar spot. The heating was discontinued and the reaction mixture was allowed to cool. Most of the acetone was evaporated under vacuo, and the remaining residue was diluted with ether (50 mL) and washed successively with water. The organic ether layer was dried over MgSO₄ and concentrated in vacuo by rotary evaporation. The crude yellow-orange oil was further purified by flash column chromatography over silica gel using hexanes to 15% hexanes:ethyl acetate gradient. The homogenous fractions from TLC were collected, combined and evaporated under reduced pressure to yield the allyl ether product (5.5 g, 95%)¹H NMR (400 MHz, CDCl₃): 4.65 (dt, J=1.6, 5.2 Hz, 2H, OCH₂), 5.39 (d, J=12.0 Hz, 1H, ═CH₂), 5.45 (d, J=18.0 Hz, 1H, ═CH₂), 5.98 (m, 1H, ═CH), 6.72 (m, 1H, ArH).

### Step 3: Preparation of (3-Allyloxy-5,6-difluoro-2-nitro-phenyl)-(2-fluoro-4-iodo-phenyl)-amine

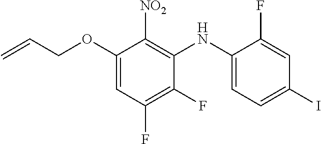 [SMILES: C=CCOc1cc(F)c(F)c(Nc2ccc(I)cc2F)c1[N+](=O)[O-]]

To a solution of 2-fluoro-4-iodo-phenylamine (1.1 g, 4.6 mmol) in THF (50 mL) was dropwise added LHMDS solution (6.0 mL, 6.0 mmol, 1 Min THF) at −78° C. After stirring for 1 h at −78° C., a solution of 1-allyloxy-3,4,5-trifluoro-2-nitrobenzene (1.2 g, 5.1 mmol) in THF (10 mL) was dropwise added into the reaction mixture. The reaction mixture was stirred at −78° C. for additional 1 hand brought to room temperature and stirred for 16 h. The progress of reaction was monitored by ¹H NMR. After completion, the solvent was removed under reduced pressure. The residue obtained was dissolved in ethyl acetate, washed with water, dried over anhydrous Na₂SO₄ and concentrated. The residue was triturated with hexane to yield (3-allyloxy-5,6-difluoro-2-nitro-phenyl)-(2-fluoro-4-iodo-phenyl)-amine as a yellow solid (900 mg). ¹H-NMR (400 MHz, CDCl₃): 4.62 (2H, d, J 4.8), 5.33-5.36 (1H, d, J=10), 5.48 (1H, d, J=17.2), 5.98-6.02 (1H, m), 6.22 (1H, dd, J=2.4, 9.6), 6.36 (1H, dd, J=2, 10.4), 7.04-7.08 (1H, m), 7.45-7.52 (2H, m), 7.79 (1H, s).

### Step 4: Preparation of 6-Allyloxy-3,4-difluoro-N2-(2-fluoro-4-iodo-phenyl)-benzene-1,2-diamine

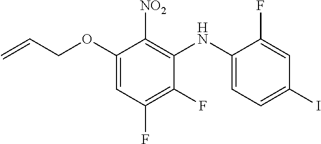 [SMILES: C=CCOc1cc(F)c(F)c(Nc2ccc(I)cc2F)c1[N+](=O)[O-]]

A suspension of (3-allyloxy-5,6-difluoro-2-nitro-phenyl)-(2-fluoro-4-iodo-phenyl)-amine (7, 0.9 g, 2 mmol) in ethanol (12 mL) was stirred at 70° C. to obtain a clear solution. To this hot solution, was added a freshly prepared solution of Na₂SO₄ (1.04 g, 6 mmol) in water (2.5 mL). The reaction mixture was stirred at 90° C. for 1 h. The progress of reaction was monitored by TLC. After completion, the solvent was removed under reduced pressure. The residue was diluted with ethyl acetate, washed with water, and the organic phase was dried over anhydrous Na₂SO₄ and concentrated to yield 6-allyloxy-3,4-difluoro-N2-(2-fluoro-4-iodo-phenyl)-benzene-1,2-diamine as a brown solid (730 mg). 1H-NMR (400 MHz, CDCl₃): 3.86 (2H, bs), 4.54 (2H, d, J=5.2), 5.34 (1H, d, J=10.8), 5.42 (1H, d, J=17.2), 5.66 (1H, bs), 6.02-6.09 (1H, m), 6.20 (1H, d, J=8.4), 6.62-6.66 (1H, m), 7.33 (1H, dd, J=2, 8.4), 7.63 (1H, d, J=2).

### Step 5: Preparation of 1-Allyl-N-(3,4-difluoro-2-(2-fluoro-4-iodophenyamino)-6-allyloxyphenyl)cyclopropane-1-sulfonamide

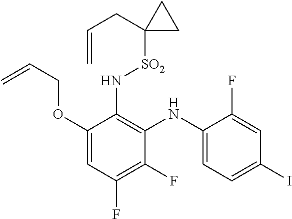 [SMILES: C=CCOc1cc(F)c(F)c(Nc2ccc(I)cc2F)c1NS(=O)(=O)C1(CC=C)CC1]

3-(Allyloxy)-5, 6-difluoro-N-(2-fluoro-4-iodophenyl) benzene-1,2-diamine (420.2 mg, 1.0 mmol) is dissolved in anhydrous pyridine (1.0 mL), and to this solution is added 1-allyl-cyclopropyl-1-sulfonyl chloride (250.0 mg, 1.38 mmol, freshly prepared) at room temperature. The mixture is heated in an oil bath under nitrogen for 48 h. The TLC analysis of mixture indicated that a new polar spot is formed when compared with starting material. The reaction mixture is diluted with ethyl acetate and washed with 0.01 M HCl, water, and brine. The organic layer is dried over MgSO₄ and concentrated under reduced pressure. Flash chromatography of crude material over silica gel using 30 to 40% hexanes:ethyl acetate affords pure compound (375 mg, 66%) MS analysis: [M+H]⁺ 565; 1H NMR (400 MHz, CDCl₃): 0.81 (t, J=6.0 Hz, 2H, Cylopropyl-CH₂), 1.26 (t, J=6.0 Hz, 2H, Cylopropyl-CH₂), 2.73 (d, J=8.0 Hz, 2H, ----CH₂), 4.62 (dt, J=1.2, 5.2 Hz, 2H, OCH₂), 5.08 (dd, J=1.2, 16.0 Hz, 1H, ═CH₂), 5.13 (dt, J=1.6, 8.0 Hz, 1H═CH₂), 5.44 (dd, J=1.6, 8.0 Hz, 1H, ═CH₂) 5.51 (dt, J=1.6, 8.0 Hz, 1H, ═CH₂), 5.69 (m, 1H, ═CH), 6.07 (m, 1H, ═CH), 6.12 (1H, s, NH), 6.44 (m, 1H, ArH), 6.56 (dd, J=4.0, 12.0 Hz, 1H, ArH), 7.28 (d, J=8.0 Hz, 1H, ArH), 7.40 (dd, J=1.0, 8.0 Hz, 2H, ArH).

### Step 6: Preparation of (Z)-10,11-difluoro-12-((2-fluoro-4-iodophenyl)amino)-4,7-dihydro-1H-spiro[benzo[b][1,5,4]oxathiazecine-3,1′-cyclopropane]2,2-dioxide

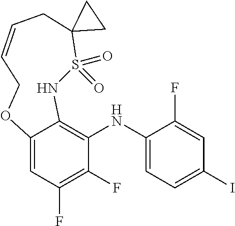 [SMILES: O=S1(=O)Nc2c(cc(F)c(F)c2Nc2ccc(I)cc2F)OC/C=C\CC12CC2]

### Method A.

A diluted solution of Zhang catalyst [prepared as described in Tetrahedron Letters 46 (2005) 7225-7228](1.5 mg/mL, 50 μL) in CH₂Cl₂ was added to a CH₂Cl₂ solution (1.0 mL) of l-Allyl-N-(3,4-difluoro-2-(2-fluoro-4-iodophenyamino)-6-allyloxyphenyl)cyclopropane-1-sulfonamide from Step 5 (6.3 mg, 0.011 mmol) at room temperature and the mixture was stirred at room temperature for an additional 24 h. The mixture was then concentrated and purified by preparative TLC (silica gel) developing with hexanes:ethyl acetate, and the band corresponding to a new compound was collected and eluted with acetone. The desired compound was isolated as a solid (4.8 mg, 80%). MS analysis: [m+H]⁺ 537; ¹H NMR (400 MHz, CDCl₃): 0.74 (brs s, CH₂), 1.14 (brs, CH₂), 3.14 (m, 2H, CH₂), 4.92 (s, 2H, OCH₂), 5.46 (dd, J=12.0 Hz, 1H, ═CH), 5.72 (dd, J=8.0, 12.0 Hz, 1H, ═CH), 6.27 (s, 1H, NH), 6.51 (m, 2H, ArH), 7.18 (s, 1H, NH), 7.29 (d, J=8.0, 1H, ArH), 7.41 (d, J=12.0 Hz, 1H, ArH).

### Method B.

To a degassed solution of bis-olefin (from Step 5, 930 mg, 1.64 mmol) in dichloroethane (60 mL), Hoveyda-Grubbs 2nd generation catalyst (120 mg, 0.19 mmol, 10 mol %) was added. The reaction mixture was stirred at 70° C. for 3 h. The progress of reaction was monitored by TLC. After completion, the reaction mixture was concentrated under reduced pressure. The residue was purified by flash column chromatography to yield the desired compound (225 mg).

### Step 7: Preparation of 10,11-difluoro-12-((2-fluoro-4-iodophenyl)amino)-5,6-dihydroxy-4,5,6,7-tetrahydro-1H-spiro[benzo[b][1,5,4]oxathiazecine-3,1′-cyclopropane]2,2-dioxide

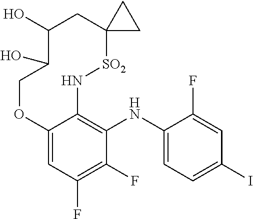 [SMILES: O=S1(=O)Nc2c(cc(F)c(F)c2Nc2ccc(I)cc2F)OCC(O)C(O)CC12CC2]

To a THF solution (0.5 mL) of (E)-10,11-difluoro-12-((2-fluoro-4-iodophenyl)amino)-4,7-dihydro-1H-spiro[benzo[b][1,5,4]oxathiazecine-3,1′-cyclopropane]2,2-dioxide (4.8 mg, 0.009 mmol) was added NMO (5.0 mg) followed by OSO₄ as a solution (5.0 μL, 4% wt in water), via syringe at room temperature. The mixture was stirred over night (14 h). Starting material is completely consumed by TLC analysis to form a highly polar product (50% hexanes:ethyl acetate). The mixture was diluted with ethyl acetate (5.0 mL), washed with Na₂S₂O₃ (1% solution, 2.0 mL), water, and finally with brine. The organic layer was separated, dried over MgSO₄ and evaporated. The crude compound was purified by preparative TLC, and the most polar band moved by ethyl acetate was collected. Extraction of the collected silica band by acetone yielded racemic diol product (3.8 mg, 74%). MS analysis: [M+H]+ 571; ¹H NMR (400 MHz, CDCl₃): 0.68 (brs s, 2H, CH₂), 0.75 (m, 1H), 1.17 (brs, 1H, CH₂), 2.07 (s, 2H, CH₂), 2.12 (s, 2H, CH₂), 3.10-2.50 (m, 3H), 3.65 (m, 1H), 3.85 (d, 1H), 4.04 (brs, 1H), 4.42 (brt, 1H), 6.40 (m, 1H), 6.88 (s, 1H, ArH), 7.28 (d, 1H, ArH), 7.30 (d, J=8.0, 1H, ArH).

Stereoisomers including Compound 1 were obtained using SFC separation under the following conditions: Hexane:Ethanol (90:10 v/v); Column: Chiralcel OD-H (250×4.6 mm) 5 uM; Flow Rate: 1.5 ml/min, Temperature: Ambient; Concentration: 1.0 mg/ml, UV Detection: 220 nm.

7.2 Attempts at Crystallization of Compound 1 using different solvents

Early crystallization experiments were performed of Compound 1.

These crystallization attempts included the following:
Slow evaporation of 10 mg of Compound 1 dissolved in 1 mL ethyl alcohol or 1 mL mixture of solvents such as ethyl alcohol and carbon tetrachloride;
Slow evaporation of 10 mg of Compound 1 dissolved in 1 mL ethyl acetate;
Slow evaporation of 10 mg of Compound 1 in a mixture of 1 mL dichloromethane and methanol; and
Dissolution of 10 mg of Compound 1 in 1 mL ethyl acetate followed by dropwise addition of n-hexane.

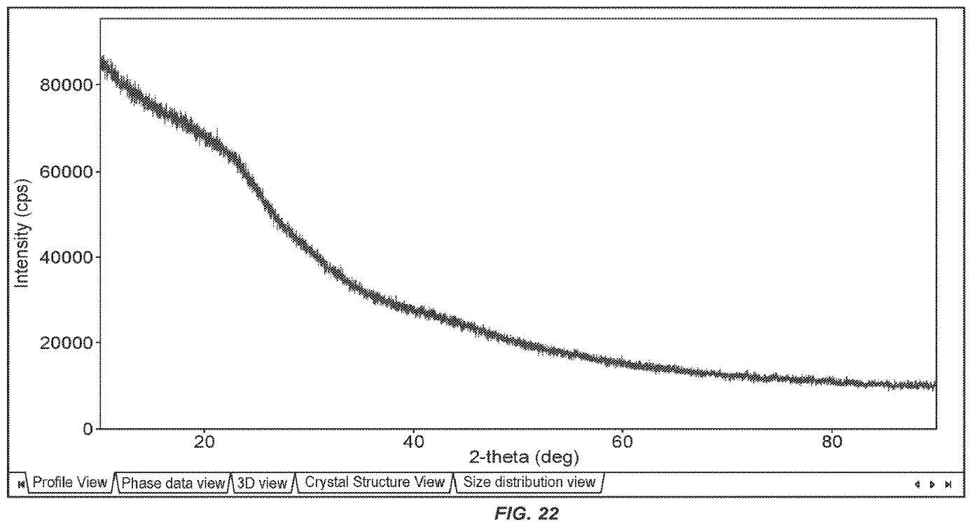

In all attempts, no crystallization of Compound 1 was observed. Only an oily substance or amorphous form was obtained from these crystallization attempts. The amorphous solid had the XRPD as shown in FIG. 22.

### 7.3 Polymorph Screening of Compound 1

### Purification of Compound 1

52 g of Compound 1 from section 6.1 above was suspended in a 500 mL of a mixture of 30% ethyl acetate/heptanes. Stirred at 70° C. (water bath temperature) using rotary evaporator for 30 minutes. After cooling to room temperature, the mixture was filtered, washed with a mixture of 30% ethyl acetate/heptanes and dried overnight under high vacuo to obtain 40 g of purified material. The LCMS of the purified material showed a purity of >98%.

### I. Summary of Identified Polymorphs and Pseudo-Polymorphs

Polymorphic behaviors of purified Compound 1 were investigated by equilibration, temperature cycling, slow cooling, fast cooling, slow evaporation, fast evaporation, anti-solvent addition, vapor diffusion and heat-cool DSC experiments. In total, about 110 experiments were conducted.

Compound 1 showed polymorphic behaviors. In total, 3 crystalline forms were identified to be polymorphs or pseudo-polymorphs of the free form (Table 1), including 1 anhydrate, named as Form A, and 2 solvates, named as Form B and C. In addition, amorphous form was obtained by fast evaporation, slow evaporation, some anti-solvent addition and vapor diffusion experiments.

TABLE 1
Summary of generation conditions of polymorphs and
pseudo-polymorphs

| Polymorphs or | |
|---|---|
| pseudo- | |
| polymorphs | Screening experiments |
| Form A, | From most of solvent systems |
| anhydrate | by equilibration. |
| | From MeOH/water by anti- |
| | solvent addition. |
| | From IPA/hexane by vapor |
| | diffusion. |
| Form B, ACN | From ACN and EtOH/ACN |
| solvate | by equilibration. |
| Form C, MeOH | From MeOH by equilibration. |
| solvate | |
| Amorphous | From most of solvent systems |
| form | by slow evaporation or fast |
| | evaporation. |
| | From IPAc/heptane or DMSO/ |
| | water by anti-solvent addition. |
| | From EtOAc/hexane by vapor |
| | diffusion. |

### II. Characterization of Polymorphs and Pseudo-Polymorphs

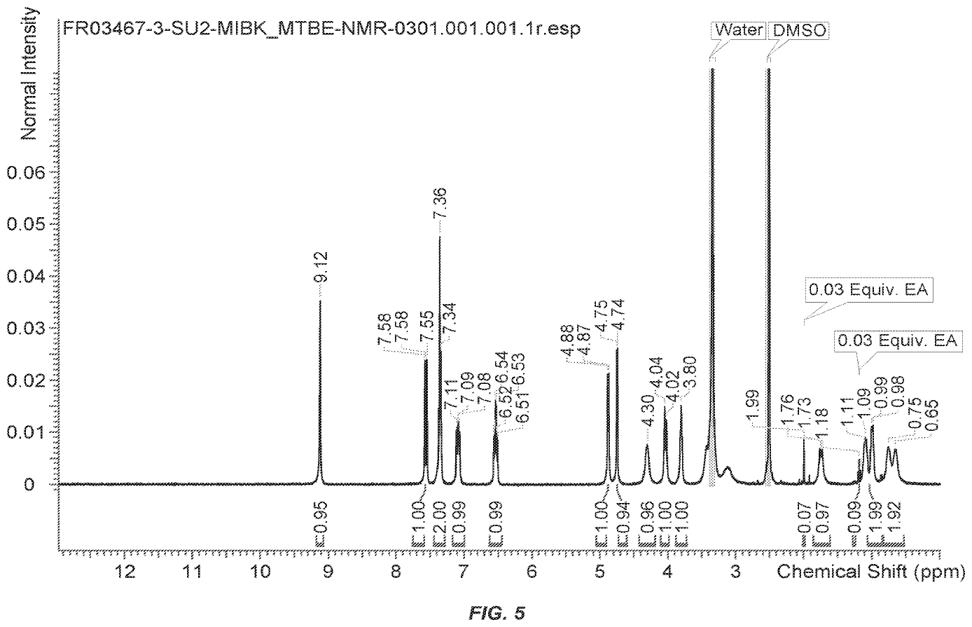

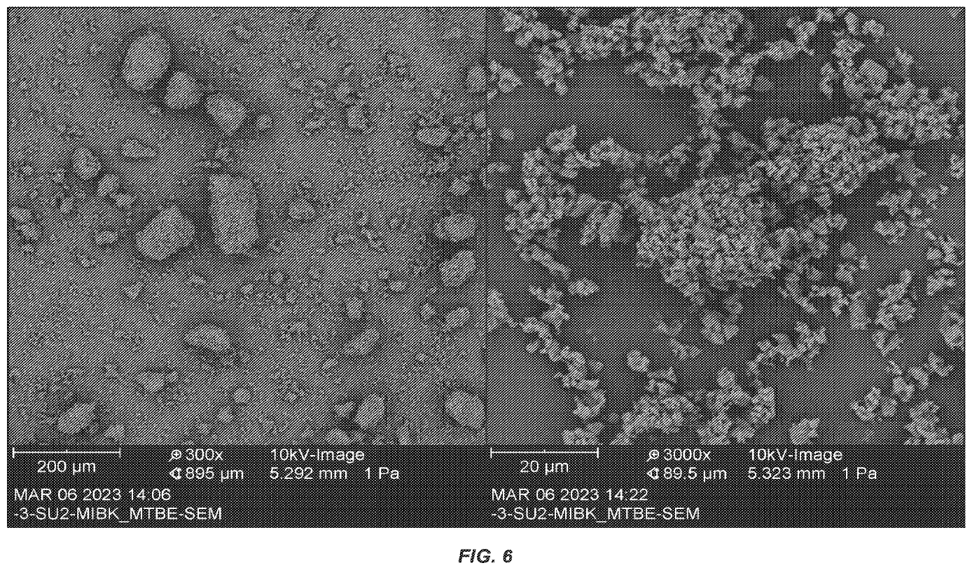

Form A is an anhydrate. It was obtained from most of solvent systems by equilibration, and from MeOH/water by anti-solvent addition experiments. It was also obtained from IPA/hexane by vapor diffusion experiment. Form A is of high crystallinity. DSC showed a melting peak at Tₒₙₛₑₜ of 189.2° C. with an enthalpy of 78 J/g. TGA shows about 0.4% weight loss at 170° C. and about 0.5% weight loss from 170° C. to 210° C. HPLC shows 99.1% purity. ¹H-NMR shows about 0.03 equiv. of EtOAc (0.5% by weight), see FIG. 5. SEM shows the sample consists of small-sized irregular particles, aggregating to up to 200 μm, see FIG. 6.

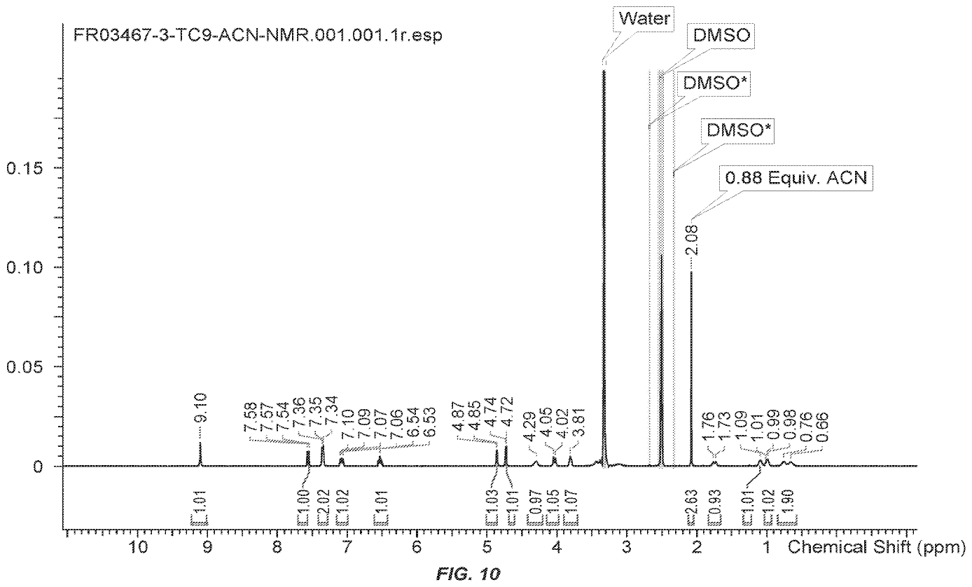

Form B is a solvate of ACN. It was obtained from ACN or EtOH/ACN by equilibration experiments. Form B is of high crystallinity. DSC showed a desolvation peak at Tₒₙₛₑₜ of 95.9° C. with an enthalpy of 24 J/g and a melting peak at Tₒₙₛₑₜ of 190.2° C. with an enthalpy of 66 J/g. TGA shows about 6.5% weight loss at 185° C. ¹H-NMR shows about 0.9 equiv. of ACN (6.1% by weight), see FIG. 10. Form B transformed to Form A after heating to 170° C. upon desolvation.

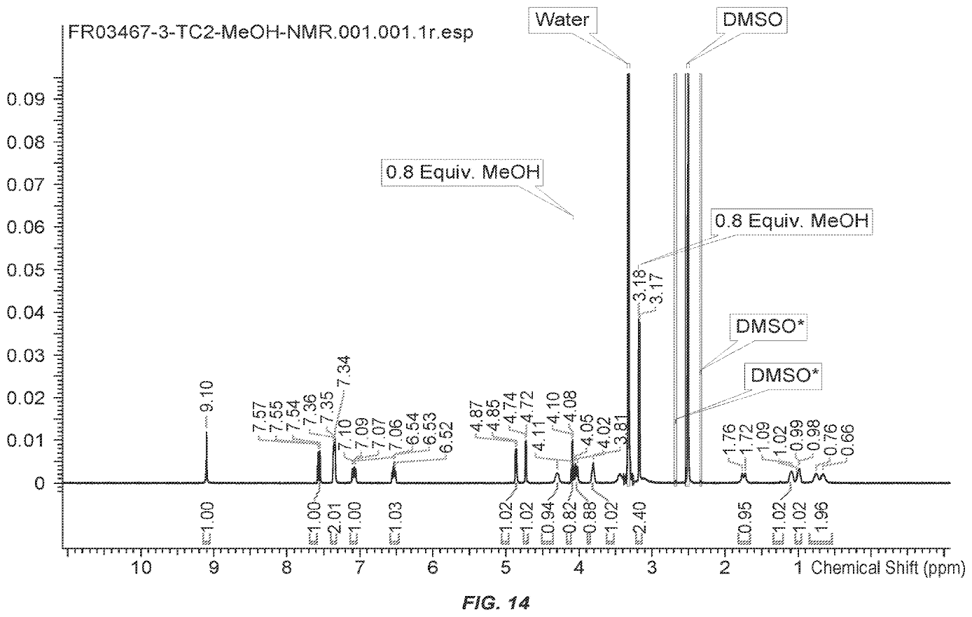

Form C is a solvate of MeOH. It was obtained from MeOH by equilibration experiment. Form C is of high crystallinity. DSC showed a desolvation peak at Tₒₙₛₑₜ of 131.6° C. with an enthalpy of 3 J/g and a melting peak at Tₒₙₛₑₜ of 191.1° C. with an enthalpy of 79 J/g. TGA shows about 3.2% weight loss at 185° C. and about 1.2% weight loss from 185° C. to 210° C. ¹H-NMR shows about 0.8 equiv. of MeOH (4.3% by weight), see FIG. 14. Form C transformed to Pattern A after heating to 185° C. upon desolvation.

The amorphous form was obtained from most of the solvent systems by fast evaporation or slow evaporation, and from EtOAc/hexane by vapor diffusion experiments. Besides, it was also obtained from IPAc/heptane or DMSO/water by anti-solvent addition experiments. Modulated DSC showed a glass transition temperature at 58.1° C. with delta Cₚ of 0.4 J/(g ° C.). TGA shows about 2.7% weight loss at 130° C. and 7.5% weight loss from 130° C. to 215° C.

TABLE 2
Summary of characterization of polymorphs and
pseudo-polymorphs

| | Form A | Form B | Form C | Amor- |
|---|---|---|---|---|
| | (anhy- | (ACN | (MeOH | phous |
| | drate) | solvate) | solvate) | form |
| Crystallinity | High | High | High | Amor- |
| (by XRPD) | | | | phous |
| | | | | form |
| Thermal | Melting | Desolvation | Desolvation | Glass |
| events (by | peak | peak | peak | transition: |
| DSC or | Tₒₙₛₑₜ: | Tₒₙₛₑₜ: | Tₒₙₛₑₜ: | 58.1° C., |
| mDSC) | 189.2ºC | 95.9° C.; | 131.6° C.; | delta Cₚ: |
| | | melting | melting | 0.4 J/(g. |
| | | peak | peak | ° C. |
| | | Tₒₙₛₑₜ: | Tₒₙₛₑₜ: | |
| | | 190.2° C. | 191.1º | |
| Enthalpy | 78 | 24, 66 | 3, 79 | |
| (by | | | | |
| DSC, | | | | |
| J/g) | | | | |
| Weight | 0.4% @ | 6.5% @ | 3.2% @ | 2.7% @ |
| loss | 170° C., | 185° C. | 185° C., | 130° C., |
| (by | 0.5% | | 1.2% | 7.5% |
| TGA) | from | | from | from |
| | 170° C. | | 185° C. | 130° C. |
| | to | | to | to |
| | 210° C. | | 210° C. | 215° C. |
| Residual | 0.03 | 0.9 equiv. | 0.8 equiv. | 0.3 equiv. |
| solvent | equiv. | ACN | MeOH | acetone |
| (by | EtOAc | (6.1% | (4.3% | (3.0% |
| ¹H-NMR) | (0.5% | by | by | by |
| | by | weight) | weight) | weight) |
| | weight) | | | |
| Morphology | Irregular | // | // | // |
| (by SEM) | particles | | | |
| | with | | | |
| | particle | | | |
| | size | | | |
| | <10 μm, | | | |
| | aggre- | | | |
| | gation | | | |
| | to up to | | | |
| | 200 μm | | | |

Explanation “//” : Not available.

### III. Evaluation of Form A

Form A was further evaluated in terms of bulk stability, solubility and hygroscopicity.

a. Solubility

Experiment 1: 5 mg of Form A and 5 mg of amorphous form were weighed into an 8 mL glass vial, respectively. 2.5 mL of solubility medium was added. Obtained suspensions/solutions were stirred at 37° C. at 400 rpm and sampled at 2 hours and at 24 hours. Obtained suspensions/solutions were filtered through a 0.45 μm PVDF membrane filter by centrifugation at 14,000 rpm and at 37° C. Filtrate were analyzed by HPLC and pH meter for solubility and pH value, respectively. Residual solids (wet cakes) from the 24 hours samples were also characterized by XRPD to determine physical form.

TABLE 3
Solubility (target concentration 2 mg/mL)
Solubility at 37° C., target concentration 2 mg/mL
(in free form), equilibration for 24 hours,
LOQ: 0.0005 mg/mL

| | Physical form | | | | | |
|---|---|---|---|---|---|---|
| | Form A | | | Amorphous form | | |
| | Solubility | | | Solubility | | |
| | (mg/mL) | | | (mg/mL) | | XRPD of |
| Solubility | | 24 h | XRPD of | | 24 h | residual |
| media | 2 h | (pH) | residual solid | 2 h | (pH) | solid |
| HCl solution, | 0.012 | 0.0037 | Form A | 0.014 | 0.013 | Form A |
| pH 1.2 | | (1.30) | | | (1.29) | |
| Acetate buffer | 0.012 | 0.0051 | Form A | 0.017 | 0.017 | Amorphous |
| (50 mM), pH 4.5 | | (4.59) | | | (4.48) | form |
| Phosphate buffer | 0.013 | 0.0051 | Form A | 0.016 | 0.016 | Amorphous |
| (50 mM), pH 6.8 | | (6.77) | | | (6.75) | form |
| FaSSGF, pH 1.6 | 0.012 | 0.0063 | Form A | 0.016 | 0.014 | Amorphous |
| | | (1.60) | | | (1.67) | form |
| FaSSIF-v1, pH 6.5 | 0.27 | 0.33 | Form A | 1.1 | 1.2 | Amorphous |
| | | (6.52) | | | (6.48) | form |
| FeSSIF-v1, pH 5.0 | 1.8 | ≥2.0 | //, very thin | >2 | >2 | // |
| | | (4.97) | suspension that may | | (5.01) | |
| | | | be caused by the | | | |
| | | | medium itself. | | | |
| Water | 0.019 | 0.013 | Form A | 0.038 | 0.034 | Amorphous |
| | | (8.17) | | | (7.66) | form |

Explanation “//”: Not carried out because no sufficient solid was obtained

Experiment 2: 5 mg of Form A and 5 mg of amorphous form were weighed into an 8 mL glass vial, respectively. 1 mL of solubility medium was added. Obtained suspensions/solutions were stirred at 37° C. at 400 rpm and sampled at 2 hours and at 24 hours. Obtained suspensions/solutions were filtered through a 0.45 μm PVDF membrane filter by centrifugation at 14,000 rpm and at 37° C. Filtrate were analyzed by HPLC and pH meter for solubility and pH value, respectively.

TABLE 4
Solubility (target concentration 5 mg/mL)

| | Solubility at 37° C., target concentration 5 mg/mL (in free | | | | | | |
|---|---|---|---|---|---|---|---|
| | form), equilibration for 24 hours, LOQ: 0.0005 mg/mL | | | | | | |
| | | Physical form | | | | | |
| | | Form A | | | Amorphous form | | |
| | | Solubility | | | Solubility | | |
| | | (mg/mL) | | XRPD of | (mg/mL) | | XRPD of |
| Exp. | Solubility | | 24 h | residual | | 24 h | residual |
| ID | media | 2 h | (pH) | solid | 2 h | (pH) | solid |
| ES8 | FeSSIF-v1, | 2.4 | 3.0 | // | >5 | >5 | // |
| | pH 5.0 | | (4.93) | | | (4.94) | |

Explanation “//”: Not carried out because no sufficient solid was obtained.

b. Bulk Stability

Form A was placed at 25° C./92.5% RH in an open container, at 40° C./75% RH in an open container, and at 60° C. in a closed container for 1 week. Samples after the stress were characterized by XRPD and HPLC and inspected for color change.

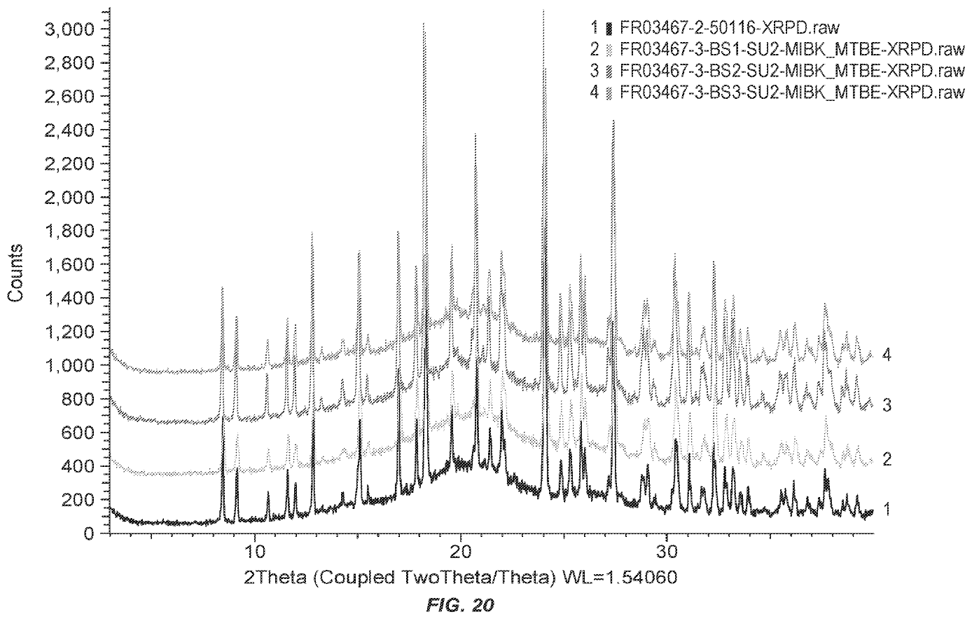

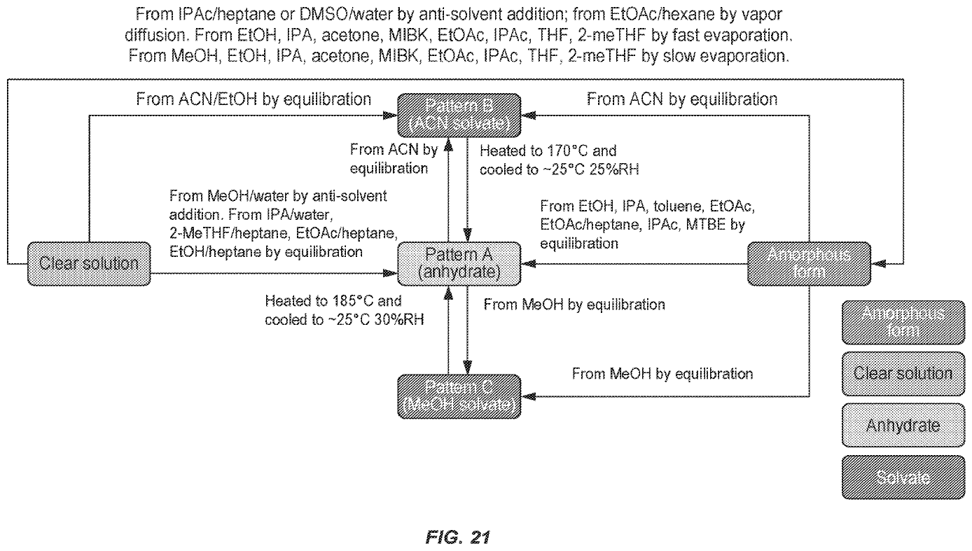

In all cases, Form A was confirmed and no change of color was observed. Results are summarized in FIG. 20.

c. Hygroscopicity

Water sorption and desorption behavior Form A was investigated by DVS at 25° C. with a cycle of 40-0-95-0-40% RH. XRPD was measured after the DVS test to determine form change. Form A is slightly hygroscopic. The DVS test measured 0.4% water uptake from 40% to 80% RH, see FIG. 4. XRPD confirmed pattern of Form A (no form change).

d. Mechanic Properties

Compression simulation experiments: about 20 mg of Form A was compressed for 5 minutes under 2 MPa, 5 MPa and 10 MPa with a hydraulic press. Potential form change and degree of crystallinity were evaluated by XRPD. No obvious decrease in crystallinity was observed in all tested conditions.

Dry grinding simulation experiments: about 20 mg of Form A was ground manually with a mortar and a pestle for 5 min. Potential form change and degree of crystallinity were evaluated by XRPD. Only a slight decrease in crystallinity was observed by showing broader peaks.

Wet granulation simulation experiments: water or ethanol was added drop-wisely to about 20 mg of Form A until the sample was wetted sufficiently. Wet sample was ground gently with in a mortar and a pestle for 5 min. Post granulation sample was dried under ambient condition for 10 min. Potential form change and degree of crystallinity were evaluated by XRPD. Only a slight decrease in crystallinity was observed by showing broader peaks.

### IV. Preparation of Polymorphs

### 1. Preparation of Form A

Form A was prepared using the procedure below:
About 300 mg of free form Pattern A was weighed into an 8 mL glass vial.
Into the vial, about 1 mL of MIBK/MTBE was added.
Resultant suspension was stirred at 50° C. with a stirring bar on a magnetic stirring plate at a rate of 400 rpm for 4 days.
Obtained suspension was filtered through a 0.45 μm nylon membrane filter by centrifugation at 14,000 rpm.
About 190 mg of free form Pattern A was obtained as light brown solids with a yield of 63.3%.

Form A was characterized as discussed herein.

### 2. Preparation of the Amorphous Form

The amorphous form was prepared using the procedure below.
About 2 g of Form A was weighed into a 20 mL glass vial.
Into the vial, about 7 mL of acetone was added.
Resultant solution was filtered through a 0.45 m nylon membrane filter by injection with a syringe and fast evaporated under a dry nitrogen flow.
The resultant solids were dried at 25° C. overnight under vacuum.
Amorphous form was obtained as brown solids.

Amorphous form was characterized as discussed herein.

### V. Phase 1 Clinical Trial of Compound 1

A Phase 1 Open-Label Study to Assess the Safety, Tolerability, Pharmacokinetics, and Pharmacodynamics of Compound 1 in Patients with MAPK Pathway-Driven Advanced Solid Tumors with a Documented RAS, NF1, or RAF Mutation or Patients Who have Failed BRAF/MEK Inhibition

The Phase 1 clinical trial is a multi-center, open-label, dose escalation 3+3 study design to evaluate the safety, tolerability, pharmacokinetic (PK), pharmacodynamic (PD), and preliminary efficacy of Compound 1 in patients with MAPK pathway driven advanced solid tumors with a documented RAS, NF or RAF mutation or patients who have failed BRAF/MEK inhibition.

Compound 1 was teasted in people with advanced solid tumors with rat sarcoma virus (RAS), neurofibromatosis type I (NF1), or rapidly accelerated fibrosarcoma (RAF) mutations.

Patients with histologically or cytologically diagnosed mitogen-activated protein kinase (MAPK) pathway driven advanced solid tumors with all of the following characteristics qualified for enrollment:
i. tumor cannot be surgically resected
ii. patient has failed or is ineligible for standard of care therapy
iii. patient has no available treatment options with known clinical benefit
iv. documented evidence of rat sarcoma virus (RAS), neurofibromatosis type I (NF1), and/or rapidly accelerated fibrosarcoma (RAF) mutations. Patients with RAF mutations must have previously failed v-Raf murine sarcoma viral oncogene homolog B (BRAF)/MEK inhibition.

Patients in the study were administered Compound 1 by mouth as a single dose, followed by one week observation, then once a day during the study, in 28-day cycles. Participants will continue on daily Compound 1 for up to 2 years, or until:
i. they decide to withdraw from the study, or
ii. they experience unacceptable side effects, or
iii. their disease progresses, or another illness interferes with taking the study drug, or
iv. the sponsors stops the study.

In this study, Compound 1 was presented in 1 mg, 4 mg, and 10 mg strength capsules, intended for oral administration once daily. Sequential dose escalation is: 2 mg, 4 mg, 8 mg, 15 mg, 22 mg, 30 mg, 37 mg, and 45 mg.

In some embodiments, the capsule formulation comprises a solid form of Compound 1 described herein.

### A. Pharmacokinetic Profile of Compound 1

### 1. Interim Phase I Results

PK data from the 2 mg and 4 mg cohorts in first-in-human Phase 1 clinical trial of Compound 1 demonstrated a PK and safety profile that differentiates Compound 1 as a next-generation MEK inhibitor. Compound 1 achieved significant exposures with a favorable safety profile without showing adverse side effects such as rash or GI toxicity, which are typical for MEK inhibitors even at low doses. The long half-life at approximately 70 hours, and the ability to achieve a flat PK curve at steady-state, aim to provide a constant target inhibition while avoiding peak plasma toxicities, which is a unique PK profile among MEK inhibitors used for the treatment of Neurofibromatosis type 1 (NF1).

TABLE 5
Parmacokinetic (PK) data for cohort 1 (2 mg) and
cohort 2 (4 mg) at day 1 and day 22 (steady state)

| Dose | Cmax | Cmin | AUC₀₋₂₄ |
|---|---|---|---|
| Cohort | (ng/mL) | (ng/mL) | (ng*h/mIL)** |
| 2 mg Day 1 | 4.91 (101.3) | N/A | 86.3 (89.2) |
| 2 mg Day 22 | 16.2 (89.7) | 10.2 (119.4) | 354 (95.2) |
| 4 mg Day 1 | 6.36 (135.6) | N/A | 100 (97.1) |
| 4 mg Day 22 | 61.3 (10.3) | 51.5 (20.8) | 1,390 (12.8) |

**Data are Geometric Mean (Geometric coefficient of variation (CV) %), Cmax: highest concentration of drug after a dose is given; Cmin: lowest concentration of a drug after a dose is given; AUC: area under the concentration-time curve and measures the total drug exposure (the extent) across time.

### 2. PK Results:

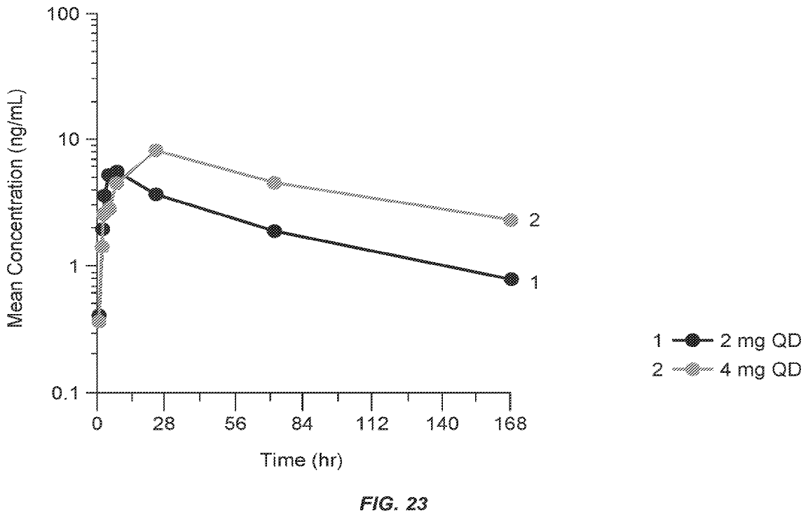

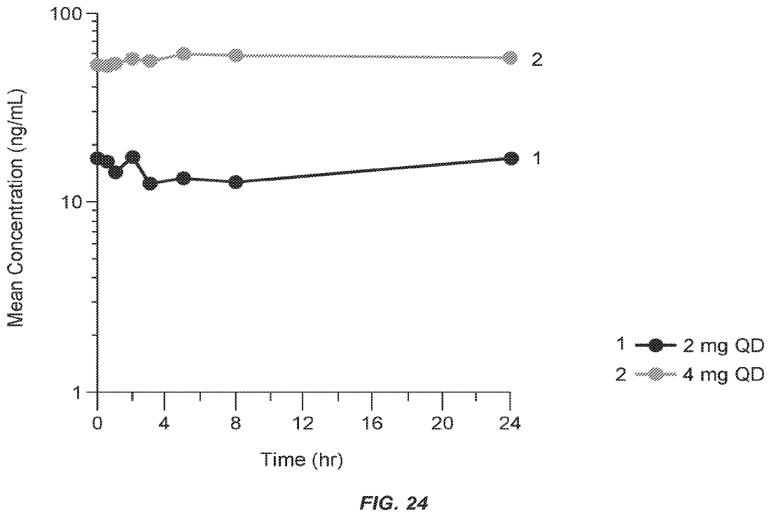

Plasma exposure increased with an increase in dose and linear PK was observed;
Long half-life of approximately 70 hours will allow for once daily dosing or longer intervals;
Prolonged systemic exposure with minimal fluctuation in Compound 1 plasma concentration at steady state (Cmax/Cmin ratio of 1.2) indicates a potential to achieve constant target inhibition. See, FIGS. 23 and 24.

At steady-state, drug levels peaked at about 5 hours with a geometric mean maximum concentration (Cmax) of 16.2 and 61.3 ng/mL for the 2 mg and 4 mg dose groups, respectively. The mean elimination half-life was 67.9 hours supporting once-daily or less frequent oral dosing.

Compound 1 has a significantly longer half-life compared to early generation MEK inhibitors, particularly those used for the treatment of NF1, which have halflives of less than 8 hours.

### 3. Safety and Tolerability

In the first 2 dosing cohorts (n=6), Compound 1 was shown to be well-tolerated with a favorable safety profile with no drug-related dose interruptions, reductions or discontinuations. There were no drug-related serious adverse events (SAE) in any dose arm and no protocol-defined stopping criteria were met. Importantly, at the 2 and 4 mg dose levels no rash or skin toxicity, gastrointestinal (GI) toxicity, or ocular toxicity have been observed to date.

### CONCLUSIONS

Unlike first-generation MEK inhibitors for the treatment of NF1 that require twice-daily dosing (BID) and exhibit short half-lives (<8 hours), Compound 1 has the potential to achieve prolonged target inhibition due to its long half-life of approximately 70 hours with once-daily dosing (QD). The PK profile showed consistent plasma levels at steady-state, as reflected by a low Cmax to Cmin ratio, potentially reducing the risks for Cmax-related toxicity. These findings supports the benefit of Compound 1 for both the treatment of NF1, particularly, cutaneous and plexiform neurofibromas in NF1, cancer and other MAPK-driven opportunities.

The embodiments provided herein are not to be limited in scope by the specific embodiments provided in the examples which are intended as illustrations of a few aspects of the provided embodiments and any embodiments that are functionally equivalent are encompassed by the present disclosure. Indeed, various modifications of the embodiments provided herein are in addition to those shown and described herein will become apparent to those skilled in the art and are intended to fall within the scope of the appended claims.

A number of references have been cited, the disclosures of which are incorporated herein by reference in their entirety.

1. A solid form comprising Compound 1:

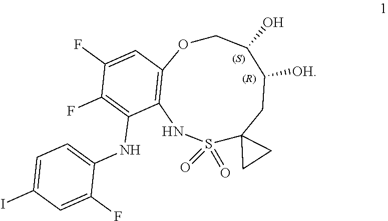

2. The solid form of claim 1, comprising a free base of Compound 1.

3. The solid form of claim 2, characterized by an XRPD pattern comprising peaks at approximately 8.5, 15.1, and 18.3° 2θ.

4. The solid form of claim 3, wherein the XRPD pattern further comprises peaks at approximately 9.2, 12.0, and 17.0° 2θ.

5. The solid form of claim 4, wherein the XRPD pattern further comprises peaks at approximately 20.8, 22.0, and 24.1, and 27.4° 2θ.

6. The solid form of claim 3, which is characterized by an XRPD pattern substantially as shown in FIG. 1A or FIG. 1B.

7. The solid form of claim 2, characterized by an XRPD pattern comprising peaks at approximately 8.4, 12.5, and 16.9° 2θ.

8. The solid form of claim 7, wherein the XRPD pattern further comprises peaks at approximately 17.6, 17.9, and 18.1° 2θ.

9. The solid form of claim 8, wherein the XRPD pattern further comprises peaks at approximately 20.4, 23.5, and 23.8° 2θ.

10. The solid form of claim 9, wherein the XRPD pattern further comprises peaks at approximately 25.2, 27.1, and 29.2° 2θ.

11. The solid form of claim 7, which is characterized by an XRPD pattern substantially as shown in FIG. 7.

12. The solid form of claim 2, characterized by an XRPD pattern comprising peaks at approximately 8.3, 12.6, and 16.6° 2θ.

13. The solid form of claim 12, wherein the XRPD pattern further comprises peaks at approximately 17.4, 18.1, and 18.4° 2θ.

14. The solid form of claim 13, wherein the XRPD pattern further comprises peaks at approximately 20.7, 23.4, and 23.6° 2θ.

15. The solid form of claim 14, wherein the XRPD pattern further comprises peaks at approximately 25.4, 26.9, 27.9, and 29.9° 2θ.

16. The solid form of claim 12, which is characterized by an XRPD pattern substantially as shown in FIG. 11.

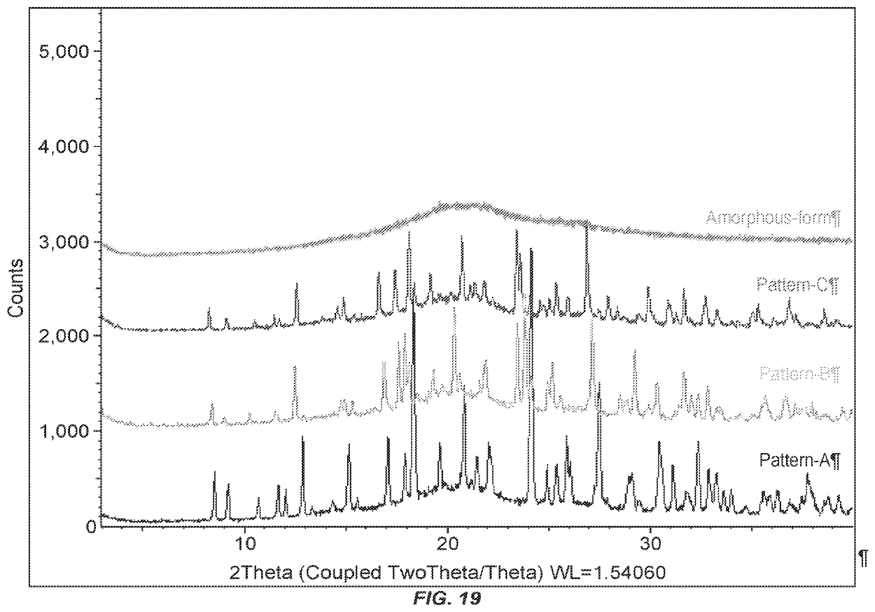

17. The solid form of claim 1, which is characterized by an XRPD pattern substantially as shown in FIG. 19.

18. The crystal form of claim 1, wherein the form contains no less than about 90%, no less than about 95%, no less than about 98%, no less than about 99%, or no less than about 99.5% by weight of one or more other crystalline form of claim 1.

19. A mixture comprising two or more crystal forms of the solid form of claim 1, wherein at least one of the two or more crystal forms are characterized by an XRPD pattern selected from the following:
i. an XRPD pattern comprising peaks at approximately 8.4, 12.5, and 16.9° 2θ; and
ii an XRPD pattern comprising peaks at approximately 8.3, 12.6, and 16.6° 2θ.

20. The crystal form of claim 1, wherein Compound 1 has a purity of >98%.

21. An amorphous solid comprising Compound 1:

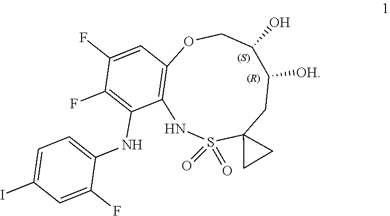

22. A pharmaceutical composition comprising a solid form of claim 1 and a pharmaceutically acceptable excipient or carrier.

23. A method of treating a disease or disorder in which the RAS pathway is upregulated comprising administering a therapeutically effective amount of of Compound 1: 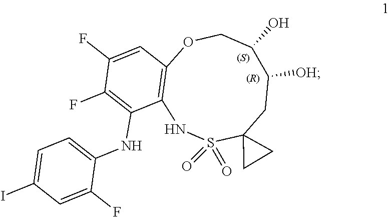
to a patient having the disease or disorder.

24. The method of claim 23, wherein the disease or disorder is a Neurofibromatosis (NF) related disorder.

25. The method of claim 23, wherein the disease or disorder is a cancer, RASopathy, or laminopathy.

26. A pharmaceutical composition comprising the amorphous solid of claim 21 and a pharmaceutically acceptable excipient or carrier.

27. A method of treating a disease or disorder in which the RAS pathway is upregulated comprising administering a therapeutically effective amount an amorphous solid comprising Compound 1: 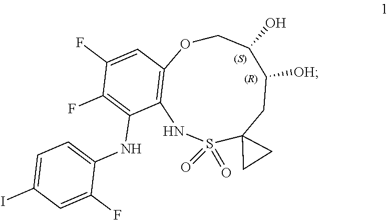
to a patient having the disease or disorder.

28. The method of claim 27, wherein the disease or disorder is a cancer, RASopathy, or laminopathy.

29. The method of claim 27, wherein the disease or disorder is a Neurofibromatosis (NF) related disorder.
